(19) United States
(12) Patent Application Publication
(10) Pub. No.: US 2009/0012079 A1
(43) Pub. Date: Jan. 8, 2009

(54) Triazolopyridine Compounds

(76) Inventors: Russell Andrew Lewthwaite, Kent (GB); John Paul Mathias, Kent (GB); Christopher Phillips, Kent (GB)

Correspondence Address: PFIZER INC., PATENT DEPARTMENT, MS8260-1611, EASTERN POINT ROAD, GROTON, CT, 06340, US

(21) Appl. No.: 12/162,018

(22) PCT Filed: Jan. 29, 2007
(86) PCT No.: PCT/IB2007/000291

§ 371 Date: Jul. 24, 2008

Related U.S. Application Data
(60) Provisional application No. 60/772,356, filed on Feb. 9, 2006.

(51) Int. Cl. A61K 31/5377; C07D 471/02; A61K 31/437; A61P 11/08; A61P 11/00; A61P 11/06; C07D 413/14

(52) U.S. Cl. 514/233.2; 546/119; 514/303; 544/127

(57) ABSTRACT

A compound of formula (Ia): or a pharmaceutically acceptable salt and/or solvate (including hydrate) thereof, or a compound of formula (Ib): or a pharmaceutically acceptable salt and/or solvate (including hydrate) thereof, and the use of a compound of formula (Ia) or (Ib) in the treatment of a TNF-mediated disease, disorder, or condition, or a p38-mediated disease, disorder, or condition, in particular the allergic and non-allergic airway diseases, more particularly obstructive or inflammatory airways diseases, preferably chronic obstructive pulmonary disease.

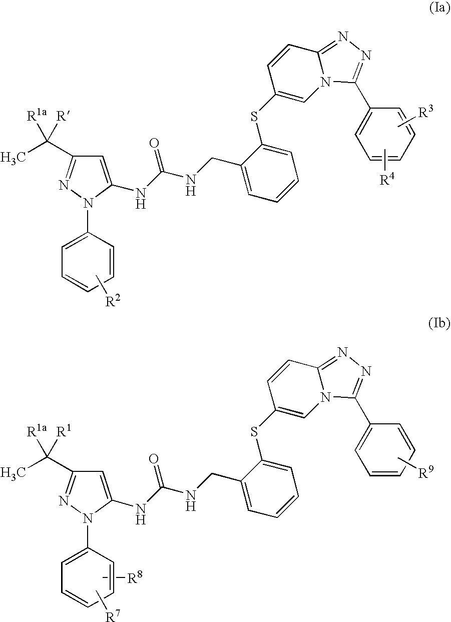

This invention relates to triazolopyridinylsulfanyl derivatives. More particularly, this invention relates to pyrazolyl-[(triazolopyridinylsulfanyl)-benzyl]-urea derivatives comprising an amino group, and to processes for the preparation of, intermediates used in the preparation of, compositions containing and the uses of, such derivatives.

The triazolopyridinylsulfanyl derivatives of the present invention are inhibitors of p38 mitogen activated protein kinase (“p38 MAPK”, “p38 kinase” or “p38”), particularly p38a kinase, and are inhibitors of tumor necrosis factor (“TNF”) production, particularly TNFα. They have a number of therapeutic applications, particularly in the treatment of allergic and non-allergic airways diseases, more particularly obstructive or inflammatory airways diseases such as chronic obstructive pulmonary disease (“COPD”).

Mitogen activated protein kinases (MAP) constitute a family of proline-directed serine/threonine kinases that activate their substrates by dual phosphorylation. The kinases are activated by a variety of signals, including nutritional and osmotic stress, UV light, growth factors, endotoxin, and inflammatory cytokines. The p38 MAP kinase group is a MAP family of various isoforms, including p38α, p38β, and p38γ. These kinases are responsible for phosphorylating and activating transcription factors (e.g., ATF2, CHOP, and MEF2C), as well as other kinases (e.g., MAPKAP-2 and MAPKAP-3). The p38 isoforms are activated by bacterial lipopolysaccharide, physical and chemical stress, and pro-inflammatory cytokines, including tumor necrosis factor (“TNF”) and interleukin-1 (“IL-1”). The products of the p38 phosphorylation mediate the production of inflammatory cytokines, including TNF.

TNF is a cytokine produced primarily by activated monocytes and macrophages. Excessive or unregulated TNF production (particularly TNF-α) has been implicated in mediating a number of diseases, and it is believed that TNF can cause or contribute to the effects of inflammation in general.

IL-8 is another pro-inflammatory cytokine, which is produced by mononuclear cells, fibroblasts, endothelial cells, and keratinocytes. This cytokine is associated with conditions including inflammation. IL-1 is produced by activated monocytes and macrophages, and is involved in inflammatory responses. IL-1 plays a role in many pathophysiological responses, including rheumatoid arthritis, fever, and reduction of bone resorption. TNF, IL-1, and IL-8 affect a wide variety of cells and tissues, and are important inflammatory mediators of a wide variety of conditions. Compounds which inhibit p38 kinase will inhibit IL-1, IL-8, and TNF synthesis in human monocytes.

P38 kinase inhibitors are well known to the person skilled in the art. J. Med. Chem. 2002, 45, 2994-3008 discloses certain pyrazole urea compounds as inhibitors of p38 kinase. International patent application PCT/IB02/00424 (WO 02/072579) discloses triazolopyridines as inhibitors of MAP kinases, preferably p38 kinase. PCT/IB2005/002574 (WO 2006/018718) relates to pyrazolyl-[(triazolopyridinylsulfanyl)-benzyl]-urea derivatives.

International patent application PCT IB2004/000363 (WO 2004/072072), publication date 26 Aug. 2004, discloses triazolopyridines useful as anti-inflammatory compounds for treating certain diseases. This is incorporated by reference in its entirety.

The compounds of the present invention are potentially useful in the treatment of a wide range of disorders. In addition to the treatment of obstructive or inflammatory airways diseases, it is believed that the compounds of the present invention can be used to treat TNF/p38 mediated diseases such as: asthma, chronic or acute bronchoconstriction, bronchitis, acute lung injury and bronchiectasis, inflammation generally (e.g. inflammatory bowel disease), arthritis, neuroinflammation, pain, fever, fibrotic diseases, pulmonary disorders and diseases (e.g., hyperoxic alveolar injury), cardiovascular diseases, post-ischemic reperfusion injury and congestive heart failure, cardiomyopathy, stroke, ischemia, reperfusion injury, renal reperfusion injury, brain edema, neurotrauma and brain trauma, neurodegenerative disorders, central nervous system disorders, liver disease and nephritis, gastrointestinal conditions, ulcerative diseases, ophthalmic diseases, opthalmological conditions, glaucoma, acute injury to the eye tissue and ocular traumas, diabetes, diabetic nephropathy, skin-related conditions, myalgias due to infection, influenza, endotoxic shock, toxic shock syndrome, autoimmune disease, graft rejection, bone resorption diseases, multiple sclerosis, psoriasis, disorders of the female reproductive system, pathological (but non-malignant) conditions, such as hemaginomas, angiofibroma of the nasopharynx, and avascular necrosis of bone, benign and malignant tumors/neoplasia including cancer, leukaemia, lymphoma, systemic lupus erthrematosis (SLE), angiogenesis including neoplasia, hemorrhage, coagulation, radiation damage, and/or metastasis. Chronic release of active TNF can cause cachexia and anorexia, and TNF can be lethal.

TNF has also been implicated in infectious diseases. These include, for example, malaria, mycobacterial infection and meningitis. These also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

The treatment of obstructive or inflammatory airways diseases is a preferred use. All forms of obstructive or inflammatory airways diseases are potentially treatable with the compounds of the present invention, in particular an obstructive or inflammatory airways disease that is a member selected from the group consisting of chronic eosinophilic pneumonia, COPD, COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

There is a need to provide new TNF inhibitors/p38 kinase inhibitors that are good drug candidates. Preferably, the new TNF inhibitors/p38 kinase inhibitors show good potency, high levels of selectivity over other related protein kinases, have properties particularly suitable for providing effective treatment via the inhalation route, are suitable for the treatment of allergic and non-allergic airways diseases (particularly obstructive or inflammatory airways diseases), are non-toxic and demonstrate few side-effects, have physical properties suitable for administration by inhalation, exist in a physical form that is stable and non-hygroscopic, and/or are easily formulated. The compounds of the present invention can form acid addition salts, by reaction of the amino substituent R² or R⁹ of compounds of formula (Ia) or (Ib), with a suitable acid. As the salt form they have solubility characteristics that are particularly suitable for a drug candidate, in addition to other desirable properties for a drug candidate. In an alternative embodiment, the free molecule has desirable solubility characteristics in addition to other desirable properties for a drug candidate.

According to one aspect of the present invention, there is provided:

a compound of formula (Ia):

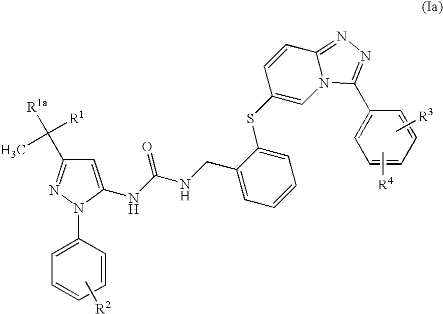

or a pharmaceutically acceptable salt and/or solvate (including hydrate) thereof, wherein

### R¹ is CH₃, SCH₃, SCH₂CH₃, CH₂CH₃, H or CH₂SCH₃;

### R^{1a} is CH₃ or CH₂CH₃;

R² is

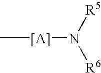

wherein A is selected from —O—(CH₂)ₓ— where x is 2 or 3, and —(CH₂)_{y}— wherein y is 1, 2 or 3;

R⁵ and R⁶ are each independently selected from methyl, ethyl and propyl, or together with the nitrogen atom to which they are attached form a pyrrolidinyl, morpholinyl, thiomorpholinyl, piperidinyl or piperazinyl ring;

and one of R³ and R⁴ is hydroxy, and the other is selected from chloro and fluoro
or a compound of formula (Ib):

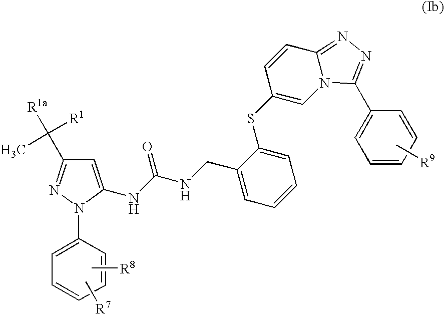

or a pharmaceutically acceptable salt and/or solvate (including hydrate) thereof, wherein
one of R⁷ and R⁸ is hydroxy, and the other is selected from chloro and fluoro;
R⁹ is in the 3 or 4 position of the phenyl ring, and is

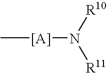

wherein A is as defined above for formula (Ia), and R¹⁰ and R¹¹ are each independently selected from methyl, ethyl, propyl, benzyl and phenylethyl, or together with the nitrogen atom to which they are attached R¹⁰ and R¹¹ form a pyrrolidinyl, piperidinyl, morpholinyl, thiomorpholinyl or piperazinyl ring, wherein said piperazinyl ring is optionally substituted at the 4 position with methyl, ethyl, propyl or benzyl, and wherein said pyrrolidinyl and piperidinyl are each optionally fused with a phenyl ring;
and R¹ and R^{1a} are as defined above for formula (Ia).

It is to be appreciated that all references herein to “treatment”, “treat” or “treating” include curative, palliative and/or prophylactic treatment.

It is to be appreciated that any references herein to a compound of formula “(I)” means “(Ia)” and/or “(Ib)”.

“Free molecule” as used herein means that the compound is not in the form of an acid addition salt formed from reaction of the amino substituent R² or R⁹ of the compound of formula (Ia) or (Ib), with an acid. The free molecule may be solvated or unsolvated.

In the salt form, the compound may be solvated or unsolvated.

“compounds of the invention” or “a compound of the invention” as used herein, unless otherwise specified, means compounds, or a compound, of formula (Ia) or formula (Ib), or a pharmaceutically acceptable salt and/or solvate thereof, and includes all polymorphs and crystal habits thereof, prodrugs and isomers thereof (including optical, geometric and tautomeric isomers), and mixtures thereof, as hereinafter defined and isotopically-labeled compounds of formula (Ia) or formula (Ib).

It has now been found that the compounds of formula (Ia) or formula (Ib), are p38 inhibitors/inhibitors of TNF production, are particularly useful for the treatment of a TNF mediated, and/or p38 mediated, disease, disorder, or condition, and are particularly suitable for administration via the inhalation route.

Preferably, R¹ is CH₃, SCH₃, CH₂SCH₃ or CH₂CH₃.

Preferably, R^{1a} is CH₃.

Preferably, A is ethoxy or methyl.

Preferably, R⁵ and R⁶ are both methyl, or together with the nitrogen atom to which they are attached form a pyrrolidinyl or a morpholinyl ring.

Preferably, R² is dimethylaminoethoxy, dimethylaminomethyl, morpholin-4-ylmethyl or pyrrolidinylmethyl.

More preferably, R² is dimethylaminoethoxy, dimethylaminomethyl or morpholin-4-ylmethyl.

Preferably, R² is in the 3-position of the phenyl ring.

Preferably, one of R³ and R⁴ is hydroxy and the other is chloro.

Preferably, R³ and R⁴ are in the 2- and 5-positions of the phenyl ring

Preferably, one of R⁷ and R⁸ is hydroxy, and the other is chloro

Preferably, R⁷ and R⁸ are in the 3- and 4-positions of the e phenyl ring.

Preferably, R⁹ is morpholin-4-ylmethyl or morpholin-4-ylethoxy.

More preferably, R⁹ is 4-(morpholin-4-ylmethyl) or 3-(morpholin-4-ylethoxy).

Preferably, R¹⁰ and R¹¹, together with the nitrogen to which they are attached form a morpholinyl ring.

In another embodiment of the invention, there is provided a compound of formula (Ia) or a pharmaceutically acceptable salt and/or solvate (including hydrate) thereof, wherein:

### R¹ is CH₃, CH₂CH₃, SCH₃, or CH₂SCH₃;

### R^{1a} is CH₃;

R² is in the 3 position and is dimethylaminoethoxy, dimethylaminomethyl, morpholin-4-ylmethyl or pyrrolidinylmethyl;
and one of R³ and R⁴ is hydroxy, and the other is selected from chloro and fluoro.

In further embodiment of the invention, there is provided a compound of formula (Ib) or a pharmaceutically acceptable salt and/or solvate (including hydrate) thereof, wherein:

### R¹ is CH₃, CH₂CH₃, SCH₃, or CH₂SCH₃;

### R^{1a} is CH₃;

R⁹ is in the 3 or 4 position of the phenyl ring, and is morpholin-4-ylmethyl or morpholin-4-ylethoxy;
and one of R⁷ and R⁸ is hydroxy, and the other is selected from chloro and fluoro.

Pharmaceutically acceptable salts of the compounds of formula (Ia) or formula (Ib), include the acid addition salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, adipate, cyclamate, tannate, pyroglutamate, naphthalene-1,5-disulfonate, xinafoate (1-hydroxynaphthalene-2-carboxylate) and trifluoroacetate salts.

In one embodiment of the invention, the compound of formula (Ia) or (Ib) is in a salt form.

Preferably, the compound of formula (Ia) or (Ib) is in a salt form, wherein the salt is selected from: acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, haphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, adipate, cyclamate, tannate, pyroglutamate, naphthalene-1,5-disulfonate, xinafoate (1-hydroxynaphthalene-2-carboxylate) and trifluoroacetate

More preferably, the compound of formula (Ia) or (Ib) is in a salt form wherein the salt is selected from acetate, mesylate, fumarate, hydrochloride/chloride, hydrobromide/bromide, bisulphate/sulphate, D-Tartrate, L-Tartrate, isethionate and xinafoate.

Hemisalts of acids may also be formed, for example, hemisulphate.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of compounds of formula (Ia) or (Ib) may be prepared by one or more of three methods:
(i) by reacting the compound of formula (Ia) or (Ib) with the desired acid;
(ii) by removing an acid—or base-labile protecting group from a suitable precursor of the compound of formula (Ia) or (Ib) or by ring-opening a suitable cyclic precursor, for example, a lactone or lactam, using the desired acid or base; or
(iii) by converting one salt of the compound of formula (Ia) or (Ib) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter, unless otherwise specified, all references to compounds of formula (Ia) or formula (Ib) include references to the free molecule, salts, solvates, hydrates and complexes thereof and to solvates and complexes of salts thereof.

The compounds of the invention include compounds of formula (Ia) or formula (Ib) as hereinbefore defined, including all polymorphs and crystal habits thereof, prodrugs and isomers thereof (including optical, geometric and tautomeric isomers) as hereinafter defined and isotopically-labeled compounds of formula (Ia) or formula (Ib).

As indicated, so-called ‘pro-drugs’ of the compounds of formula (Ia) or formula (Ib) are also within the scope of the invention. Thus certain derivatives of compounds of formula (Ia) or formula (Ib) which may have little or no pharmacological activity themselves can, when administered into or onto the body, be converted into compounds of formula (Ia) or formula (Ib) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (Ia) or formula (Ib) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (Ia) or formula (Ib) contains an alcohol functionality (—OH), an ether thereof, for example, a compound wherein the hydrogen of the alcohol functionality of the compound of formula (Ia) or formula (Ib) is replaced by (C₁-C₆)alkanoyloxymethyl; and
(ii) where the compound of formula (Ia) or formula (Ib) contains a primary or secondary amino functionality (—NH₂ or —NHR where R≠H), an amide thereof, for example, a compound wherein, as the case may be, one or both hydrogens of the amino functionality of the compound of formula (Ia) or formula (Ib) is/are replaced by (C₁-C₁₀)alkanoyl.

Further examples of replacement groups in accordance with the foregoing examples and examples of other prodrug types may be found in the aforementioned references.

Moreover, certain compounds of formula (Ia) or formula (Ib) may themselves act as prodrugs of other compounds of formula (Ia) or formula (Ib).

Also included within the scope of the invention are metabolites of compounds of formula (Ia) or formula (Ib), that is, compounds formed in vivo upon administration of the drug. Some examples of metabolites in accordance with the invention include
(i) where the compound of formula (Ia) or formula (Ib) contains a (C₁-C₆)alkyl group, the hydroxy(C₁-C₆)alkyl derivative thereof. For example where the compound of formula (Ia) or formula (Ib) contains a methyl group, the hydroxymethyl derivative thereof (—CH₃->-CH₂OH);
(ii) where the compound of formula (Ia) or formula (Ib) contains an alkoxy group, an hydroxy derivative thereof (—OR—>—OH);
(iii) where the compound of formula (Ia) or formula (Ib) contains a tertiary amino group, a secondary amino derivative thereof (—NR⁵R⁶—>—NHR⁵ or —NHR⁶);
(iv) where the compound of formula (Ia) or formula (Ib) contains a secondary amino group, a primary derivative thereof (—NHR⁵—>—NH₂);
(v) where the compound of formula (Ia) or formula (Ib) contains a phenyl moiety, a phenol derivative thereof (-Ph->-PhOH);
(vi) where the compound of formula (Ia) or formula (Ib) contains an amide group, a carboxylic acid derivative thereof (—CONH₂->COOH); and
(vii) where the compound of formula (Ia) or formula (Ib) contains a S—(C₁-C₆)alkyl group, the S(O)(C₁-C₆)alkyl derivative thereof. For example, where the compound of formula (Ia) or formula (Ib) contains a S-methyl group, the S(O)methyl derivative thereof, and where the compound formula (Ia) or formula (Ib) contains an alkyl-5-alkyl group, the alkyl-S(O)-alkyl derivative thereof.

In another aspect of the invention there is provided the active metabolites of the compounds of formula (Ia) or formula (Ib).

Compounds of formula (Ia) or formula (Ib) containing one or more asymmetric car bon atoms can exist as two or more stereoisomers. Where structural isomers are interconvertible via a low energy barrier, tautomeric isomerism (‘tautomerism’) can occur. This can take the form of proton tautomerism in compounds of formula (Ia) or (Ib) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (Ia) or formula (Ib), including compounds exhibiting more than one type of isomerism, and mixtures of one or more thereof. Also included are acid addition salts wherein the counterion is optically active, for example, d-lactate or 1-lysine, or racemic, for example, dl-tartrate or dl-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (Ia) or formula (Ib) contains a basic moiety, an acid such as tartaric acid. The resulting diastereomeric mixture may be separated by chromatography and/or fractional crystallization, and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

The present invention includes all pharmaceutically acceptable isotopically-labelled compounds of formula (Ia) or formula (Ib) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and 3H, carbon, such as ¹¹C, ¹³C and ¹⁴C, chlorine, such as ³⁶Cl, fluorine, such as ¹⁸F, nitrogen, such as ¹³N and ¹⁵N, oxygen, such as ¹⁵O, ¹⁷O and ¹⁸O, and sulphur, such as ³⁵S.

Certain isotopically-labelled compounds of formula (Ia) or formula (Ib), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (Ia) or formula (Ib) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are novel intermediates as herein defined, all salts, solvates and complexes thereof and all solvates and complexes of salts thereof as defined herein for compounds of formula (Ia) or formula (Ib). The invention includes all polymorphs of the aforementioned species and crystal habits thereof.

When preparing compounds of formula (Ia) or formula (Ib) in accordance with the invention, it is open to a person skilled in the art to routinely select the form of intermediate compound which provides the best combination of features for this purpose. Such features include the melting point, solubility, processability and yield of the intermediate form and the resulting ease with which the product may be purified on isolation.

Compounds of formula (Ia) or formula (Ib) may be prepared, in a known manner, in a variety of ways. The following routes illustrate such ways of preparing these compounds; the skilled man will appreciate that other routes may be equally as practicable. In the following routes, the substituents R²′ and R³′ refer to the corresponding substituted phenyl substituents of the compound s of formula (Ia) or formula (Ib):

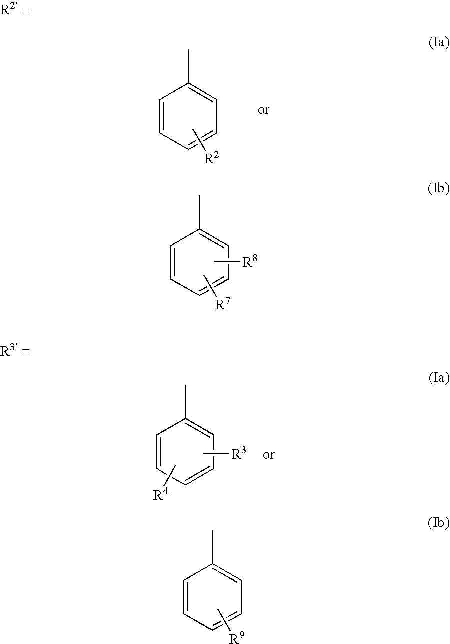

“PdCl₂(dppf). CH₂Cl₂” is 1,1-bis(diphenylphosphino)ferrocene palladium (II) chloride 1:1 dichloromethane complex

“DBU” is 1,8-diazabicyclo[5.4.0]undec-7-ene

“BOC” means tert-butoxycarbonyl;

“CBz” means benzyloxycarbonyl

“Et” means ethyl

“Me” means methyl

“Pd” means palladium, and

“eq” means mole equivalent(s)

“iPr” means isopropyl.

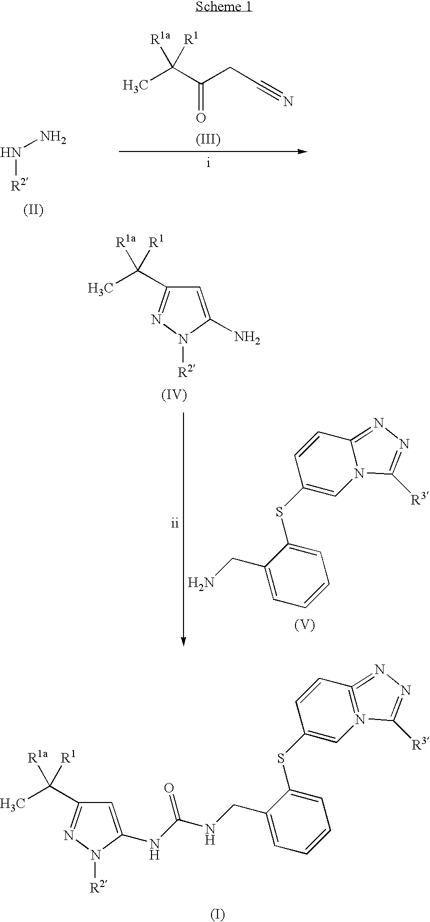

Compounds of general formula (II) are either commercially available or can be prepared as shown in scheme 2.

Compounds of general formula (III) are either commercially available (e.g. when R^{1a}=Me and R¹=Me) or can be prepared as shown in scheme 3.

Compounds of general formula (IV) can be prepared from compounds of formula (II) and (III) by process step i-cyclocondensation of compound (II) and compound (III) optionally in the presence of a suitable acid catalyst such as hydrochloric acid, optionally in the presence of a suitable base such as Hünig's base, triethylamine or pyridine, in a suitable solvent such as methanol or ethanol, at elevated temperature for 3-24 hours. Typical conditions comprise of 1.0-1.3 equivalents of compound (II) and 1.0-1.1 equivalents of compound (III) in the presence of hydrochloric acid, in ethanol, heated under reflux for 3-24 hours.

Additionally, compounds of general formula (IV) can be obtained by direct condensation of compounds of formula (VII) with compounds of formula (III), in EtOH/HCl.

Compounds of general formula (IV) can also be obtained using conditions found in J. Org. Chem. 2004, 69, 5578-5587.

Compounds of general formula (V) can be prepared as shown in scheme 4.

Compounds of formula (I) can be prepared from compounds (IV) and (V) by process step ii—urea formation is achieved by reaction of compound (IV) in the presence of a suitable carbonyl source such as N,N′-carbonyldiimidazole, phenylchloroformate or bis(trichloromethyl) carbonate and a suitable base such as Hünig's base or pyridine, in a suitable solvent such as dichloromethane, THF (tetrahydrofuran) or 1,4 dioxane, under ambient conditions for 48 hours, followed by addition of compound (V). Typical conditions comprise of either:
a) 1.0 equivalent of compound (IV) and 5.0-6.0 equivalents of N,N′-carbonyldiimidazole in dichloromethane, under ambient conditions for 24 hours,
b) 0.25-0.80 equivalents of compound (V), 0.25-1.25 equivalents of Hünig's base in dichloromethane or 1,4 dioxane, under ambient conditions for 24 hours, or
c) 1 equivalent of compound (IV) and 1 equivalent of phenylchloroformate in THF/pyridine, followed by 0.8-1 equivalent of compound (V) in DMSO.

Compounds of general formula (II) may be prepared as shown in scheme 2.

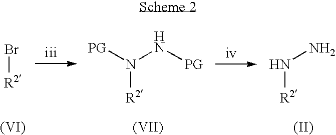

Compounds of general formula (VI) are commercially available.

The compound of formula (II) could also be prepared from the corresponding aniline derivative by diazotisation followed by reduction, using conditions well-known in the chemical literature.

PG is a suitable protecting group such as BOC or CBz and preferably BOC.

Where R²′ is, or includes, a phenol, the skilled person will appreciate that it may be necessary to use a protecting group, typically benzyloxy or methyloxy.

Compounds of general formula (II) can be prepared from compounds of general formula (VI), via compound (VII), by process steps (iii) and (iv).

Step (iii)—is achieved by formation of a suitable organometallic reagent e.g. arylMgBr, heteroarylMgBr, arylLi, or heteroarylLi, optionally prepared in situ under standard Grignard conditions or by reaction with a suitable alkyl lithium, e.g. ⁿBuLi, in a suitable solvent such as tetrahydrofuran or diethyl ether, at a temperature between −100° C. to 25° C., for 1-18 hours. The intermediate compound (VII) is formed by subsequent nucleophilic attack of a suitably protected diazocarboxylate compound, preferably di-tert-butyldiazocarboxylate, by arylMgBr/heteroary/MgBr/arylLi/heteroarylLi, in a suitable solvent such as tetrahydrofuran or diethyl ether, at −78° C. for 0.5-1.0 hours.

Step (iv)—Deprotection of compound (VII) using standard methodology as described in “Protecting Groups in Organic Synthesis” by T. W. Greene and P. Wutz. When PG=BOC, typical conditions involve saturation of intermediate (VII) with a suitable acid such as hydrochloric acid or trifluoroacetic acid, in a suitable solvent such as isopropyl alcohol, 1,4-dioxane or diethyl ether, under ambient conditions for 2-18 hours.

Compounds of general formula (III) may be prepared according to schemes 3.1 and 3.2.

When R¹=—(CH₂)ₙSR^{b}, compounds of formula (III) can be prepared as shown in scheme 3.1.

R^{b} represents methyl or ethyl.

n represents 0 or 1.

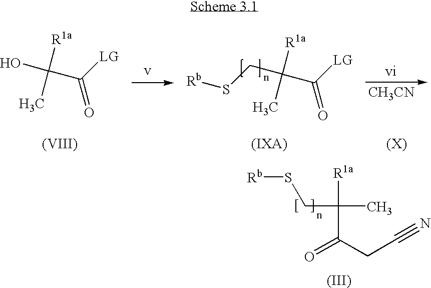

LG is a suitable leaving group, e.g. OR′ or Cl and is preferably OR′.

R′ represents C₁-C₄alkyl, and preferably C₁-C₂ alkyl.

When R′=Et or Me, compounds of formula (VIII) are commercially available.

When n=1, compounds of formula (IXA) can be prepared from compounds of formula (VIII) by process step v—nucleophilic substitution. The reaction proceeds via the formation of an intermediate containing a suitable leaving group LG′, such as mesylate or tosylate by reaction of compound (VIII) with mesyl chloride/anhydride or tosyl chloride, in the presence of a suitable base such as Hünig's base, triethylamine or pyridine, in a suitable solvent such as dichloromethane or diethyl ether, at low temperature for 1-2 hours. Concentration in vacuo is followed by the addition 1,4-dioxane or toluene and methanethiol sodium salt, heating under reflux for 24 hours. Typical conditions comprise of
a) 1.0 eq of compound (VIII), 1.0-1.2 eq of Hünig's base, and 1.1 eq of me thane sulfonyl chloride in dichloromethane, at 0° C. for 1-2 hours.
b) 1.1 eq methanethiol sodium salt in 1,4-dioxane, heating under reflux for 24 hours.

When n=0, compounds of formula (IXA) are commercially available

Compound (III) can be prepared from compounds of formula (IXA) by process step vi—reaction with acetonitrile (X). Treatment of (X) with a suitable base such as sodium hydride or lithium diisopropylamide, followed by quench of the intermediate anion with compound (IXA), in a suitable solvent such as tetrahydrofuran, at elevated temperature for 3 hours provides compounds of formula (III). Typical conditions comprise of 1.3 eq acetonitrile, 1.3 eq sodium hydride (60% dispersion in mineral oil) and 1.0 equivalent of compound (IXA) in tetrahydrofuran, heated under reflux for 3 hours.

When R^{1a} represents CH₃ or CH₂CH₃, compounds of formula (III) may be prepared as shown in scheme 3.2.

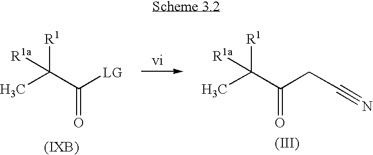

LG is a suitable leaving group, e.g. OR′ or Cl and is preferably OR′.

R′ represents C₁-C₄ alkyl, and preferably C₁-C₂ alkyl.

Compounds of formula (III) may be prepared from compounds of formula (IXB) by process step vi, as described previously.

Compounds of formula (IXB) are either available commercially, or may be prepared by analogy with the methods of Julia et. al. Bull. Soc. Chim. Fr. 1996; 133(1); 15-24, or Chuit et. al. Tetrahedron 1980; 36(16), 2305-10.

Compounds of formula (V) may be prepared as shown in scheme 4

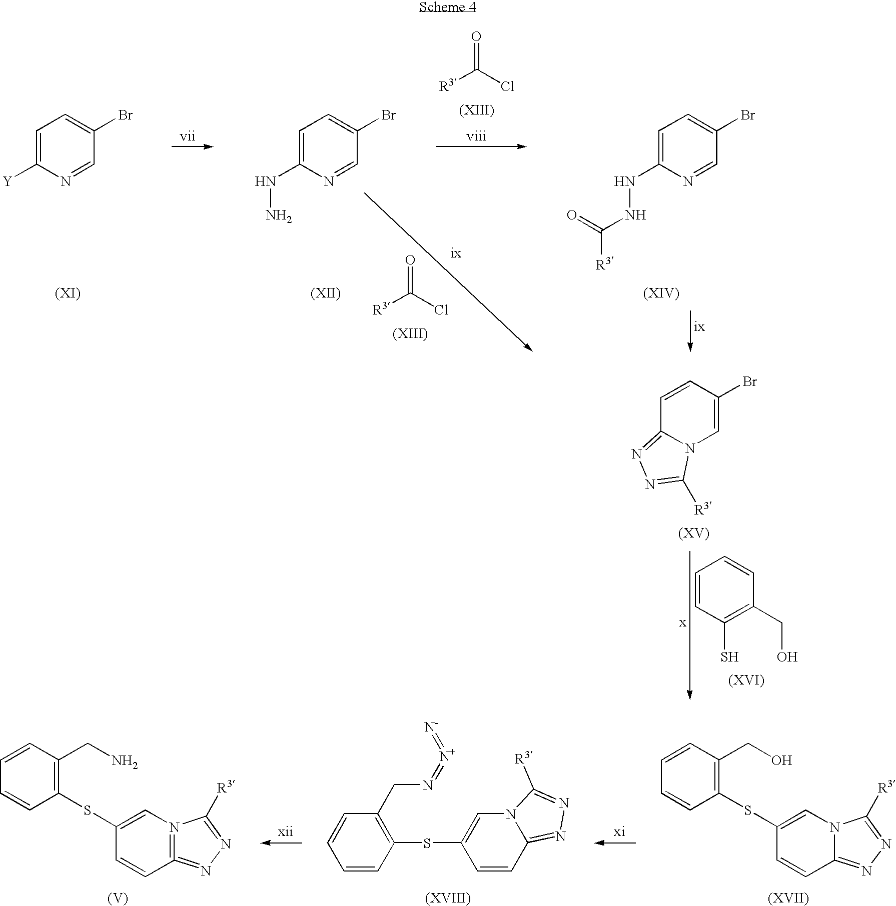

When Y=halogen and is preferably bromo, compounds of general formula (XI) are commercially available.

Compounds of formula (XII) can be prepared from compounds of formula (XI) by process step vii—reaction with hydrazine monohydrate, optionally in a suitable solvent such as methanol or ethanol, at elevated temperature for 18-72 hours. Typical conditions comprise 1.0 eq of compound (XI) and an excess of hydrazine monohydrate heated to 70° C. for 72 hours.

Compounds of formula (XIV) can be prepared from compounds of formula (XII) by process step viii-reaction with a suitable aroyl chloride R³OC(O)Cl (XIII), in the presence of a suitable base such as Hünig's base, triethylamine or pyridine in a suitable solvent such as dichloromethane or diethyl ether, at low temperature for 1-2 hours. Typical conditions comprise of 1.0 eq of compound (XII), 1.0 eq of R³′OC(O)Cl (XIII) and 5.0 eq Hünig's base in dichloromethane, at a temperature between 0-5° C. for 1-2 hours.

Compounds of formula (XV) can be prepared from compounds of formula (XIV) by process step ix—cyclisation. This is achieved by use of a suitable dehydrating agent such as phosphorus oxychloride or phosphorus (V) oxide in sulfuric acid, at elevated temperature for 18-24 hours. Typical conditions comprise of 1.0 equivalent of compound (XIV) in an excess of phosphorus oxychloride, at 75° C. for 18-24 hours.

Alternatively, compounds of formula (XV) can be prepared directly from compounds of formula (XII) by process step ix. This cyclisation is achieved by reaction with an excess of compound (XIII) and heated, for example at 95° C., for 18-24 hours.

Compounds of formula (XVII) can be prepared from compounds of formula (XV) by process step x—Pd catalysed cross coupling reaction with 2-mercaptobenzyl alcohol (XVI), in the presence of a suitable catalyst such as PdCl₂(dppfg.CH₂Cl₂, in the presence of a suitable base such as cesium carbonate or potassium carbonate, in a suitable solvent such as N,N-dimethylformamide or 1,4-dioxane, at elevated temperature for 2-48 hours. Typical conditions comprise of 1.0 eq compound (XV), 1.2-1.4 eq cesium carbonate, 1.3 eq 2-mercaptobenzyl alcohol (XVI) and 0.1 eq PdCl₂(dppf).CH₂Cl₂ in N,N-dimethylformamide, at elevated temperature for 18 hours.

Compounds of formula (XVIII) can be prepared from compounds of formula (XVII) by process step xi—azide formation. This proceeds by reaction of compound (XVII) with a suitable base such as DBU or sodium hydride, followed by reaction with a suitable azide such as diphenylphosphoryl azide in a suitable solvent such as toluene or tetrahydrofuran, at a temperature between 0-25° C. for 18-24 hours. Typical conditions comprise of 1.0 eq of compound (XVII), 1.2 eq of DBU and 1.2 eq diphenylphosphoryl azide in toluene at 0-25° C. for 24 hours.

Compounds of formula (V) can be prepared from compounds of formula (XVIII) by process step xii—reduction of compound (XVIII) with a suitable reducing agent such as triphenyl phosphine/water, tin chloride or catalytic hydrogenation, in a suitable solvent such as tetrahydrofuran or ethanol, between ambient and elevated temperature. Typical conditions comprise of 1.0 eq compound (XVIII), 1.2 eq triphenylphosphine and 1.2 eq of water in tetrahydrofuran, at room temperature for 40 hours and at 50° C. for 5 hours.

Alternatively, compounds of formula (V) can also be prepared as shown in scheme 5

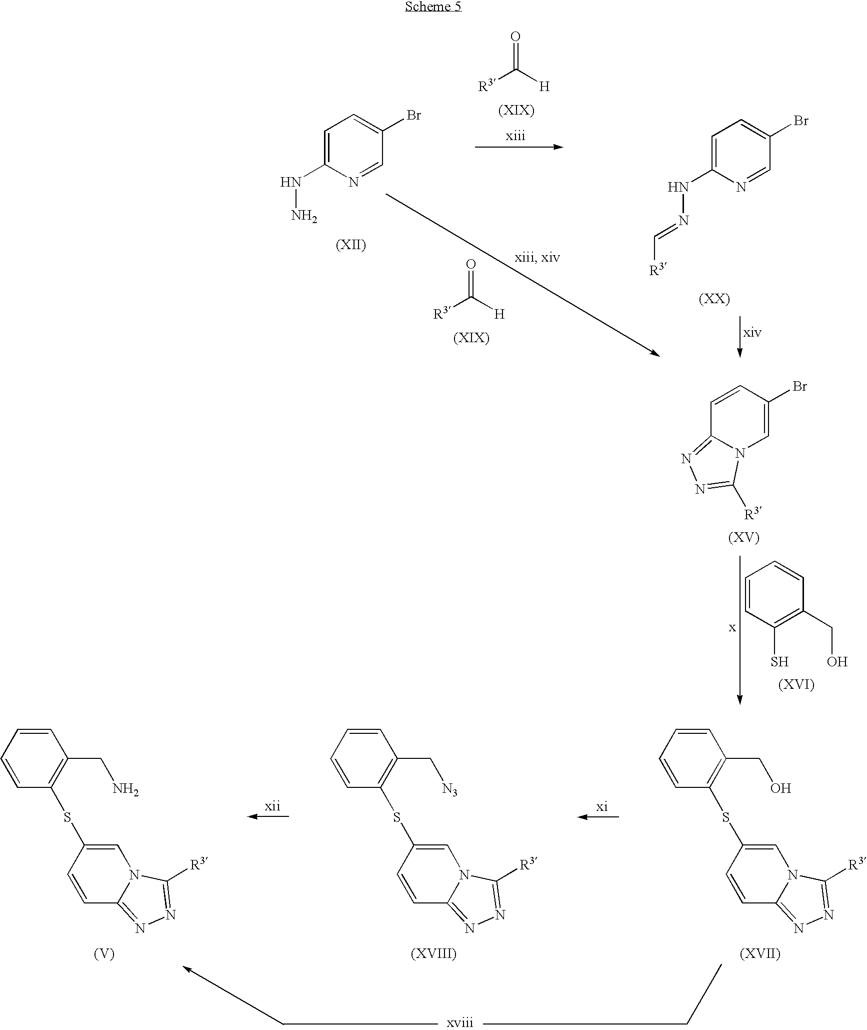

Compounds of formula (XII) can be prepared as described in scheme 4.

Compounds of formula (XIX) are either commercially available or can be prepared as described in scheme 6

Compounds of formula (XX) can be prepared from compounds of formula (XII) and (XIX) by process step xiii—condensation of hydrazine (XII) and aldehyde (XIX) in a suitable solvent such as methanol, ethanol or toluene, at elevated temperature for 0.5-1 hour. Typical conditions comprise of 1 eq of compound (XII) and 1 eq of compound (XIX) in ethanol, heated at reflux for 0.5-1.0 hour.

Compounds of formula (XV) can be prepared from compounds of formula (XX) by process step xiv—cyclisation of compound (XX) in the presence of a suitable oxidising agent such as (diacetoxyiodo)benzene, cerium (IV) ammonium nitrate or 2,3-dichloro-5,6-dicyano-1,4-benzoquinone in a suitable solvent such as ethyl acetate, dichloromethane or acetonitrile, under ambient conditions for 18-24 hours. Typical conditions comprise of 1.0 eq of compound (XX) and 1.2 eq of (diacetoxyiodo)benzene in dichloromethane, at room temperature for 24 hours.

Alternatively, compounds of formula (XV) can be prepared from compound (XII) by process steps xiii and xiv in a one-pot synthesis. Typical conditions comprise of 1 eq of compound (XII) and 1 eq of compound (XIX) in ethanol, heated at reflux for 0.5-1.0 hour, followed by addition of 1.2 eq of (diacetoxyiodo)benzene and dichloromethane, at room temperature for 24 hours.

Compounds of formula (XVII) can be prepared from compounds of formula (XV) and (XVI) by process step x as described in scheme 4.

Compounds of formula (XVIII) can be prepared from compounds of formula (XVII) by process step xi as described in scheme 4.

Compounds of formula (V) can be prepared from compounds of formula (XVIII) by process step xii as described in scheme 4.

Alternatively, compounds of formula (V) can be also be prepared from compounds of formula (XVII) by process step xviii—The reaction proceeds via the formation of an intermediate containing a suitable leaving group such as mesylate or tosylate by reaction of compound (VIII) with mesyl chloridelanhydride or tosyl chloride, in the presence of a suitable base such as Hünig's base, triethylamine or pyridine, in a suitable solvent such as dichloromethane or diethyl ether, at low to ambient temperature for 1-4 hours. The resulting intermediate is then treated with a suitable source of ammonia, typically 7M ammonia in methanol, under ambient conditions for 18-72 hours. Typical conditions comprise of 1.0 eq of compound (XVII), 3.0-4.0 eq of Hünig's base, and 2.0-3.0 eq of methane sulfonyl anhydride in dichloromethane, at 25° C. for 1-4 hours. Excess 7M ammonia in methanol is added and reaction is stirred at ambient temperature for 18-72 hours.

Alternatively-compounds of formula (V) can be prepared from compounds of formula (XV) and compound of formula (XXVII) where PG is a protecting group, such as BOC. Typical conditions comprise of 1 eq of compound (XV), 1.2 eq of compound (XXVII), 1.2 eq of anhydrous cesium carbonate, 3 eq of cesium fluoride, 0.1 eq of PdCl₂(dppf.CH₂Cl₂ in dimethylformamide as solvent at 80-100° C. for 2-48 h. The product of this reaction is then subject to acid-mediated removal of the BOC group to afford compounds of formula (V).

Compounds of formula (XXVII) can be prepared from compounds of formula (XXVIII) by process step xix (Scheme 5.1). The reaction proceeds by a palladium-catalysed insertion of the sulfide into an aromatic-bromine bond.

Typical conditions comprise of 1 eq of compound (XXVIII), 1 eq of potassium tri(isopropyl)silylsulfide (formed from 1 eq of potassium tert-butoxide and 1 eq of triisopropylsilanethiol in toluene), 1 eq of PdCl₂(dppf).CH₂Cl₂ in toluene as solvent at 100° C. for 0.5 to 2 h.

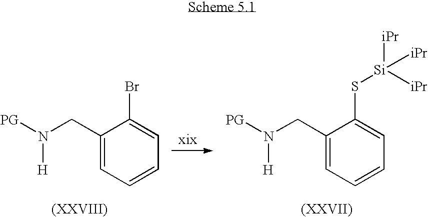

Where R³′ is, or includes, a phenol, the skilled person will appreciate that it may be necessary to use a protecting group, typically benzyloxy or methyloxy.

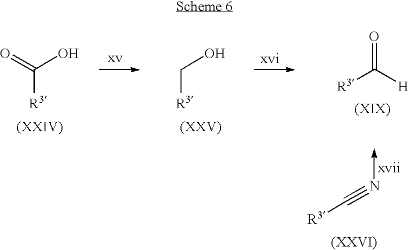

Compounds of formula (XXIV) are commercially available

Compounds of formula (XXV) can be prepared from compounds of formula (XXIV) by process step xv—reduction with a suitable reducing agent such as lithium aluminium hydride or borane in a suitable solvent such as tetrahydrofuran or dichloromethane, at elevated temperature for 6-18 hours. Typical conditions comprise of 1.0 eq of compound (XXIV) and 1.0-1.2 eq of lithium aluminium hydride in tetrahydrofuran, at reflux for 6 hours.

Compounds of formula (XIX) can be prepared from compounds of formula (XXV) by process step xvi—oxidation with a suitable oxidising agent such as manganese dioxide, potassium permanganate or oxalyl chloride/dimethylsulfoxide, in a suitable solvent such as acetone, dichloromethane or dimethylsulfoxide, at from −80 to +80° C. for 3-18 hours. Typical conditions comprise of 1.0 eq of compound (XXV) and 0.5 eq of manganese dioxide in acetone, heated under reflux for 3 hours.

Alternatively, compounds of formula (XIX) can be prepared from commercial compounds of formula (XXVI) by process step xvii—reduction of nitrile by diisobutylaluminium hydride in a suitable solvent such as tetrahydrofuran, at low temperature. Typical conditions comprise of
a) 1.0 equivalent of compound (XXVI) and 1.0-2.0 equivalents of diisobutylaluminium hydride in tetrahydrofuran, at −78° C. for 1 hour,
b) excess hydrochloric acid and water at 0° C.

It will be appreciated by those skilled in the art that it may be necessary or desirable at any stage in the synthesis of compounds of formula (Ia) or formula (Ib) to protect one or more sensitive groups in the molecule so as to prevent undesirable side reactions. In particular, it may be necessary or desirable to protect phenol groups. The protecting groups used in the preparation of compounds of formula (Ia) or formula (Ib) may be used in a conventional manner. See, for example, those described in ‘Protective Groups in Organic Synthesis’ by Theodora W Green and Peter G M Wuts, third edition, (John Wiley and Sons, 1999), in particular chapter 2, pages 17-245 (“Protection for the Hydroxyl Group”). Alternatively, the protected phenols are available commercially. Removal of such groups can be achieved using conventional methods.

It will be still further appreciated that compounds of formula (Ia) or formula (Ib) may also be converted to alternative compounds of formula (Ia) or formula (Ib) using standard chemical reactions and transformations.

In another embodiment of the invention, there is provided a process for making a corn pound of formula (Ia) or formula (Ib), wherein the substituents are as defined in claim 1 and the description related to the processes, which comprises the steps:

i: cyclocondensation of a compound of formula (II) and a compound of formula (III) to make a compound of formula (IV):

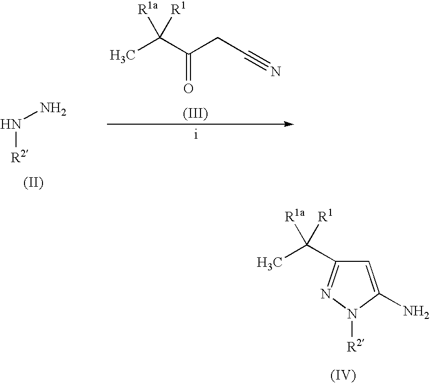

and/or
ii: urea formation, by reaction of a compound of formula (IV) with a compound of formula (V), in the presence of a suitable carbonyl source.

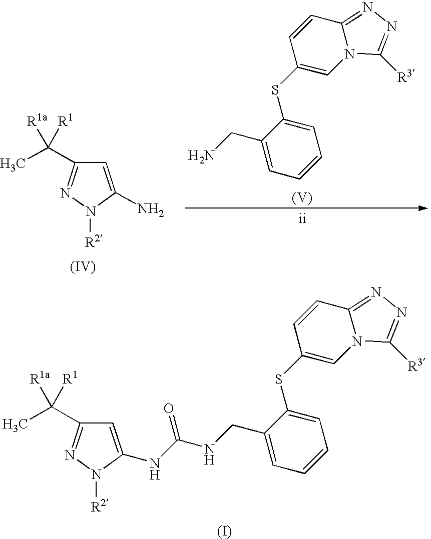

In another embodiment of the invention, there is provided a process for making a compound of formula (V), wherein the substituents are as defined in the description related to the processes, which comprises the steps:

xi: azide formation, by reaction of a compound of formula (XVII), with a suitable base, followed by reaction with a suitable azide, to form a compound of formula (XVIII)

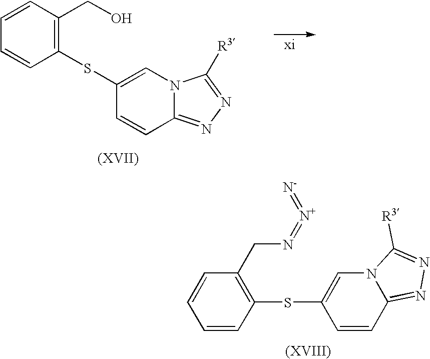

and/or
xii: reduction of a compound of formula (XVIII) to form a compound of formula (V)

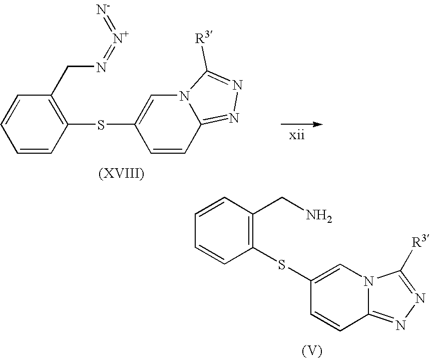

In another embodiment of the invention, there is provided a novel process as described herein.

In another embodiment of the invention, there is provided an intermediate compound of formula (IV), (V), (XVII) or (XVIII), wherein the substituents are as described herein.

In another embodiment of the invention, there is provided a novel intermediate compound of a formula as described herein.

Another aspect of the invention is a compound of formula (Ia) or formula (Ib) as described herein, or a salt and/or solvate thereof, for use in medicine.

Another aspect of the invention is a corn pound of formula (Ia) or formula (Ib) as described herein, or a salt and/or solvate thereof, for use in treating a disease, disorder, or condition selected from the group consisting of:
1. asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis,
2. chronic or acute bronchoconstriction, chronic bronchitis, small airways obstruction, and emphysema,
3. obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension,
4. bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
5. acute lung injury,
6. bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis.

A further aspect of the invention is the use of a compound of formula (Ia) or formula (Ib) as described herein, or a salt and/or solvate thereof, in the manufacture of a medicament for the treatment of a disease, disorder, or condition disclosed in paragraphs 1-6 above.

A further aspect of the invention is the use of a compound of formula (Ia) or formula (Ib) as described herein, or a salt and/or solvate thereof, in the manufacture of a medicament for the treatment of a p38-mediated disease, disorder or condition or a TNF-mediated disease, disorder, or condition.

Another aspect of the invention is a compound of formula (Ia) or formula (Ib) as described herein, or a salt and/or solvate thereof, for use in treating a p38-mediated disease, disorder or condition or a TNF-mediated disease, disorder, or condition.

The present invention provides a method of treating a mammal, including a human being, with an effective amount of a compound of formula (Ia) or formula (Ib), or a pharmaceutically acceptable salt or solvate thereof.

More precisely, the present invention provides a method of treating a p38-mediated disease, disorder or condition or a TNF-mediated disease, disorder, or condition in a mammal, including a human being, in particular a disease disorder, or condition listed above, comprising administering said mammal with an effective amount of a compound of formula (Ia) or formula (Ib), or a salt and/or solvate thereof.

Preferably, the present invention provides a compound of formula (Ia) or formula (Ib), or a pharmaceutically acceptable salt or solvate thereof, for use in treating obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension, or asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

More preferably, the present invention provides a compound of formula (Ia) or formula (Ib), or a pharmaceutically acceptable salt or solvate thereof, for use in treating chronic obstructive pulmonary disease (COPD).

Preferably, the present invention provides the use of a compound of formula (Ia) or formula (Ib), or a pharmaceutically acceptable salt or solvate thereof, in the manufacture of a medicament for treating obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension, or asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

More preferably, the present invention provides the use of a compound of formula (Ia) or formula (Ib) or a pharmaceutically acceptable salt or solvate thereof, in the manufacture of a medicament for treating chronic obstructive pulmonary disease (COPD).

As used herein, the term “TNF-mediated disease”, or “TNF-mediated disorder” or “TNF-mediated condition” refers to any disease, disorder, or condition (particularly any pathological conditions), respectively, in which TNF plays a role, either by control of TNF itself, or by TNF causing another monokine to be released, such as, for example, IL-1, IL-6, and/or IL-8. A disease state in which, for instance, IL-1 is a major component and whose production or action is exacerbated or secreted in response to TNF, would therefore be considered a disorder mediated by TNF.

As used herein, the term “p38-mediated disease”, or “p38-mediated disorder” or “p38-mediated condition” refers to any disease, disorder, or condition (particularly any pathological conditions), respectively, in which p38 plays a role, either by control of p38 itself, or by p38 causing another monokine to be released, such as, for example, IL-1, IL-6, and/or IL-8. A disease state in which, for instance, IL-1 is a major component and whose production or action is exacerbated or secreted in response to p38, would therefore be considered a disorder mediated by p38.

The compounds of the invention can be used in the treatment of a TNF-mediated disease, disorder, or condition, or a p38-mediated disease, disorder or condition, in particular the allergic and non-allergic airways diseases disclosed above, but also in the treatment of p38- or TNF-mediated conditions such as:

(a) inflammation;
(b) arthritis, such as rheumatoid arthritis, spondyloarthropathies, gouty arthritis, osteoarthritis, systemic lupus erythematosus arthritis, juvenile arthritis, osteoarthritis, and gouty arthritis;
(c) neuroinflammation;
(d) pain (i.e., use of the compounds as analgesics), such as neuropathic pain;
(e) fever (i.e., use of the compounds as antipyretics);
(f) pulmonary sarcoisosis, and silicosis;
(g) cardiovascular diseases, such as atherosclerosis, myocardial infarction (such as post-myocardial infarction indications), thrombosis, congestive heart failure, cardiac reperfusion injury, and complications associated with hypertension and/or heart failure such as vascular organ damage;
(h) cardiomyopathy;
(i) stroke, such as ischemic and hemorrhagic stroke;
(j) ischemia, such as brain ischemia and ischemia resulting from cardiac/coronary bypass;
(k) reperfusion injury;
(l) renal reperfusion injury;
(m) brain edema;
(n) neurotrauma and brain trauma, such as closed head injury;
(o) neurodegenerative disorders;
(p) central nervous system disorders (these include, for example, disorders having an inflammatory or apoptotic component), such as Alzheimer's disease, Parkinson's disease, Huntington's Disease, amyotrophic lateral sclerosis, spinal cord in jury, and peripheral neuropathy;
(q) liver disease and nephritis;
(r) gastrointestinal conditions, such as inflammatory bowel disease, Crohn's disease, gastritis, irritable bowel syndrome, and ulcerative colitis;
(s) ulcerative diseases, such as gastric ulcer;
(t) ophthalmic diseases, such as retinitis, retinopathies (such as diabetic retinopathy), uveitis, ocular photophobia, nonglaucomatous optic nerve atrophy, and age-related macular degeneration (ARMD) (such as ARMD-atrophicform);
(u) opthalmological conditions, such as corneal graft rejection, ocular neovascularization, retinal neovascularization (such as neovascularization following injury or infection), and retrolental fibroplasia;
(v) glaucoma, such as primary open angle glaucoma (POAG), juvenile onset primary open-angle glaucoma, angle-closure glaucoma, pseudoexfoliative glaucoma, anterior ischemic optic neuropathy (AlON), ocular hypertension, Reiger's syndrome, normal tension glaucoma, neovascular glaucoma, ocular inflammation, and corticosteroid-induced glaucoma;
(w) acute injury to the eye tissue and ocular traumas, such as post-traumatic glaucoma, traumatic optic neuropathy, and central retinal artery occlusion (CRAO);
(x) diabetes;
(y) diabetic nephropathy;
(z) skin-related conditions, such as psoriasis, eczema, burns, dermatitis, keloid formation, scar tissue formation, and angiogenic disorders;
(aa) viral and bacterial infections, such as sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, rhinovirus infections, and herpes virus;
(bb) myalgias due to infection;
(cc) influenza;
(dd) endotoxic shock;
(ee) toxic shock syndrome;
(fff) autoimmune disease, such as graft vs. host reaction and allograft rejections;
(gg) bone resorption diseases, such as osteoporosis;
(hh) multiple sclerosis;
(ii) disorders of the female reproductive system, such as endometriosis;
(jj) pathological, but non-malignant, conditions, such as hemaginomas (such as infantile hemaginomas), angiofibroma of the nasopharynx, and avascular necrosis of bone;
(kk) benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer, skin cancer such as squamus cell and basal cell cancers, prostate cancer, renal cell carcinoma, and other known cancers that affect epithelial cells throughout the body;
(ll) leukemia;
(mm) lymphoma, such as B cell lymphoma;
(nn) systemic lupus erthrematosis (SLE);
(oo) angiogenesis including neoplasia;
(pp) metastasis;
(qq) a fibrotic disease;
(rr) hemorrhage;
(ss) coagulation;
(tt) acute phase responses like those seen with infections and sepsis and during shock (e.g., (uu) septic shock, hemodynamic shock, etc.);
(vv) anorexia;
(ww) mycobacterial infection;
(xx) pseudorabies,
(yy) rhinotracheitis,

### (zz) HIV,

(aaa) influenza virus,
(bbb) herpes virus, including herpes simplex virus type-1 (HSV-1), herpes simplex virus type-2 (HSV-2),
(ccc) cytomegalovirus (CMV),
(ddd) varicella-zoster virus (VZV),
(eee) Epstein-Barr virus,
(fff) human herpesvirus-6 (HHV-6),
(ggg) human herpesvirus-7 (HHV-7), human herpesvirus-8 (HHV-8).

In another embodiment of the invention, there is a compound of formula (Ia) or formula (Ib), or a salt and/or solvate thereof, for use in treating a disease, disorder, or condition, selected from the list (a) to (ggg) above.

A further embodiment of the invention is the use of a compound of formula (Ia) or formula (Ib), or a salt and/or solvate thereof, in the manufacture of a medicament for treating a disease, disorder, or condition selected from the list (a) to (ggg) above.

A yet further embodiment of the invention is a method of treating a disease, disorder, or condition selected from the list (a) to (ggg) above, in a mammal, including a human being, comprising administering said mammal with an effective amount of a compound of formula (Ia) or formula (Ib), or a salt and/or solvate thereof.

The compounds of the invention can also be used in the treatment of a p38- or TNF-mediated disease such as smoke-induced airway inflammation, inflammation enhanced cough, for the control of myogenesis, for treating mucin overproduction, and/or for treating mucus hypersecretion.

As TNF-β has close structural homology with TNF-α (also known as cachectin), and because each induces similar biologic responses and binds to the same cellular receptor, the synthesis of both TNF-α and TNF-β tend to be inhibited by the compounds of this invention and thus are herein referred to collectively as “TNF” unless specifically delineated otherwise.

A compound of formula (Ia) or formula (Ib), or a pharmaceutically acceptable salt and/or solvate thereof, as mentioned above, can be administered according to the invention to animals, preferably to mammals, and in particular to humans, as pharmaceuticals

The compound can be administered per se, in a mixture with one or more other compounds of the invention, or in the form of pharmaceutical preparation, which, as active constituent contains an efficacious dose of at least one compound of the invention, in addition to customary pharmaceutically innocuous excipients and/or additives.

The compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded be fore tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of the invention, a film-forming polymer, a binder, a solvent, a humectant, a plasticiser, a stabiliser or emulsifier, a viscosity-modifying agent and a solvent. Some components of the formulation may perform more than one function.

The compounds of the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compounds of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations in dude delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

The compounds of the invention may also be administered topically to the skin or mucosa, that is, dermally or transdermally.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intra nasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of the invention. The overall daily dose will typically be in the range 0.001 mg to 40 mg which may be administered in a single dose or, more usually, as divided doses throughout the day.

In another embodiment of the invention, the compounds of the invention are preferably administered by inhalation. More preferably, the compounds of the invention are administered by inhalation with a dry powder inhaler or a metered dose inhaler, most preferably with a dry powder inhaler.

The compounds of the invention may be administered rectally or vaginally, for example, in the form of a suppository, pessary, or enema.

The compounds of the invention may also be administered directly to the eye or ear, typically in the form of drops of a micronised suspension or solution in isotonic, pH-adjusted, sterile saline.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

In another embodiment of the invention, there is provided a pharmaceutical composition comprising a compound of formula (Ia) or formula (Ib), or a salt and/or solvate thereof, and a pharmaceutically acceptable diluent, carrier or adjuvant.

In another aspect of the invention, there is provided a kit, including:
a. a compound of formula (Ia) or formula (Ib), or a salt and/or solvate thereof,
b. instructions for treating an obstructive or inflammatory airways disease, and
c. packaging for containing a and b.

Preferably, the obstructive or inflammatory airways disease is COPD.

In an alternative embodiment, the instructions in b. are for treating asthma.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus another aspect of the invention is a kit comprising two or more separate pharmaceutical compositions, at least one of which contains a compound of the invention in accordance with the invention, and means for separately retaining said compositions, such as a container, divided bottle, or divided foil packet. An example of such a kit is the familiar blister pack used for the packaging of tablets, capsules and the like.

The kit of the invention may be particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.01 mg to 10 mg depending, of course, on the mode of administration. For example, an inhaled daily dose may only require from 0.01 mg to 5 mg. The total daily dose may be administered in single or divided doses and may, at the physician's discretion, fall outside of the typical range given herein.

These dosages are based on an average human subject having a weight of about 65 kg to 70 kg. The physician will readily be able to determine doses for subjects whose weight falls outside this range, such as infants and the elderly.

According to another embodiment of the present invention, the compounds of the invention can also be used as a combination with one or more additional therapeutic agents to be co-administered to a patient to obtain some particularly desired therapeutic end result such as the treatment of pathophysiologically-relevant disease processes including, but not limited to (i) bronchoconstriction, (ii) inflammation, (iii) allergy, (iv) tissue destruction, (v) signs and symptoms such as breathlessness, cough. The second and more additional therapeutic agents may also be a compound of the invention, or one or more TNF inhibitors and/or p38 inhibitors known in the art. More typically, the second and more therapeutic agents will be selected from a different class of therapeutic agents.

As used herein, the terms “co-administration”, “co-administered” and “in combination with”, referring to the compounds of the invention and one or more other therapeutic agents, is intended to mean, and does refer to and include the following:
simultaneous administration of such combination of compound(s) of the invention) and therapeutic agent(s) to a patient in need of treatment, when such components are formulated together into a single dosage form which releases said components at substantially the same time to said patient,
substantially simultaneous administration of such combination of compound(s) of the invention and therapeutic agent(s) to a patient in need of treatment, when such components are formulated apart from each other into separate dosage forms which are taken at substantially the same time by said patient, whereupon said components are released at substantially the same time to said patient,
sequential administration of such combination compound(s) of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of compound(s) of the invention and therapeutic agent(s) to a patient in need of treatment, when such components are formulated together into a single dosage form which releases said components in a controlled, manner whereup on they are concurrently, consecutively, and/or overlappingly administered at the same and/or different times by said patient, where each part may be administered by either the same or different route.

Suitable examples of other therapeutic agents which may be used in combination with the compound(s) of the invention, or pharmaceutically acceptable salts, solvates or compositions thereof, include, but are by no means limited to:

(a) 5-Lipoxygenase (5-LO) inhibitors or 5-lipoxygenase activating protein (FLAP) a ntagonists,
(b) Leukotriene antagonists (LTRAs) including antagonists of LTB₄, LTC₄, LTD₄, and LTE₄,
(c) Histamine receptor antagonists including H1 and H3 antagonists,
(d) α₁- and α₂-adrenoceptor agonist vasoconstrictor sympathomimetic agents for decongestant use,
(e) muscarinic M3 receptor antagonists or anticholinergic agents,
(f) PDE inhibitors, e.g. PDE3, PDE4 and PDE5 inhibitors,

### (g) Theophylline,

(h) Sodium cromoglycate,
(i) COX inhibitors both non-selective and selective COX-1 or COX-2 inhibitors (NSAIDs),
(j) Oral and inhaled glucocorticosteroids, such as DAGR (dissociated agonists of the corticoid receptor)
(k) Monoclonal antibodies active against endogenous inflammatory entities,
(l) β2 agonists, including long-acting β2 agonists
(m) Adhesion molecule inhibitors including VLA-4 antagonists,
(n) Kinin-B₁- and B₂-receptor antagonists,
(o) Immunosuppressive agents,
(p) Inhibitors of matrix metalloproteases (MMPs),
(q) Tachykinin NK₁, NK₂ and NK₃ receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFκB pathway, e.g. IKK inhibitors,
(w) modulators of cytokine signalling pathways such as syk kinase, or JAK kinase inhibitors,
(x) Agents that can be classed as mucolytics or anti-tussive, and

### (y) Antibiotics.

According to the present invention, combination of the compounds of the invention with
H3 antagonists,
Muscarinic M3 receptor antagonists,
PDE4 inhibitors,
glucocorticosteroids,
Adenosine A2a receptor agonists,
β2 agonists
Modulators of cytokine signalling pathways such as syk kinase, or,
Leukotriene antagonists (LTRAs) including antagonists of LTB₄, LTC₄, LTD₄, and LTE₄, are preferred.

According to the present invention, combination of the compounds of the invention with
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate and mometasone furoate monohydrate,
muscarinic M3 receptor antagonists or anticholinergic agents including in particular ipratropium salts, namely ipratropium bromide, tiotropium salts, namely tiotropium bromide, oxitropium salts, namely oxitropium bromide, perenzepine, and telenzepine,
or β₂ agonists, in particular long-acting β2 agonists, including salmeterol, formoterol, QAB-149 and CHF-4226.
   are further preferred.

Preferably, the compounds of the invention exhibit slow-offset binding kinetics to p38.

In another preferred embodiment, when the compounds are administered via the inhalation route, they are rapidly metabolised when they have moved out of the lung.

More preferably, the compounds of the invention are metabolised to compounds that are less active than the compound administered.

In another embodiment of the invention there is provided a compound, use, method or composition, substantially as described herein.

### Assay: TNFα Screen

The anti-inflammatory properties of the compounds of the invention are demonstrated by their ability to inhibit TNFα release from human peripheral blood mononuclear cells. Venous blood is collected from healthy volunteers and the mononuclear cells purified by centrifugation through Histopaque (Ficoll) cushions. TNFα production from these cells is stimulated by addition of lipopolysaccharide. After 18 hours incubation in the presence of LPS, the cell supernatant is removed and the concentration of TNFα in the supernatant determined by ELISA. Addition of the compounds of the invention reduces the amount of TNFα produced. An IC₅₀ is determined which is equal to the concentration of compound that gives 50% inhibition of TNFα production as compared to the LPS stimulated control wells.

### p38 Kinase Assay:

Cloning of Human p38a:

The coding region of the human p38a cDNA was obtained by PCR-amplification from RNA isolated from the human monocyte cell line THP. 1. First strand cDNA was synthesized from total RNA as follows: 2 μg of RNA was annealed to 100 ng of random hexamer primers in a 10 μl reaction by heating to 70° C.: for 10 minutes followed by 2 minutes on ice. cDNA was then synthesized by adding 1 μl of RNAsin (Promega, Madison Wis.), 2 μl of 50 mM dNTP's, 4 μl of 5× buffer, 2 μl of 100 mM DTT and 1 μl (200 U) of Superscript II™ AMV reverse transcriptase. Random primer, dNTP's and Superscript II™ reagents were all purchased from Life-Technologies, Gaithersburg, Mass. The reaction was incubated at 42° C. for 1 hour. Amplification of p38 cDNA was performed by aliquoting 5 μl of the reverse transcriptase reaction into a 100 μl PCR reaction containing the following: 80 μl dH.sub.2 O, 2 μl 50 mM dNTP's, 1 μl each of forward and reverse primers (50 μmol/μl), 10 μl of 10× buffer and 1 μl Expand™ polymerase (Boehringer Mannheim). The PCR primers incorporated Bam HI sites onto the 5′ and 3′ end of the amplified fragment, and were purchased from Genosys. The sequences of the forward and reverse primers were 5′-GATCGAGGATTCATGTCTCAGGAGAGGCCCA-3′ and 5′GATCGAGGATTCT CAGGACTCCATCTCTTC-3′ respectively. The PCR amplification was carried out in a DNA Thermal Cycler (Perkin Elmer) by repeating 30 cycles of 94° C. for 1 minute, 60° C. for 1 minute and 68° C. for 2 minutes. After amplification, excess primers and unincorporated dNTP's were removed from the amplified fragment with a Wizard™ PCR prep (Promega) and digested with Bam HI (New England Biolabs). The Bam HI digested fragment was ligated into BamHI digested pGEX 2T plasmid DNA (PharmaciaBiotech) using T-4 DNA ligase (New England Biolabs) as described by T. Maniatis, Molecular Cloning: A Laboratory Manual, 2nd ed. (1989). The ligation reaction was transformed into chemically competent E. coli DH10B cells purchased from Life-Technologies following the manufacturer's instructions. Plasmid DNA was isolated from the resulting bacterial colonies using a Promega Wizard™ miniprep kit. Plasmids containing the appropriate Bam HI fragment were sequenced in a DNA Thermal Cycler (Perkin Elmer) with Prism™ (Applied Biosystems In c.). cDNA clones were identified that coded for both human p38a isoforms (Lee et al. Nature 372, 739). One of the clones that contained the cDNA for p38a-2 (CSB-2) inserted in the cloning site of PGEX 2T, 3′ of the GST coding region was designated pMON 358 02. The sequence obtained for this clone is an exact match of the cDNA clone reported by Lee et al. This expression plasmid allows for the production of a GST-p38a fusion protein.

Expression of human p38a

GST/p38a fusion protein was expressed from the plasmid pMON 35802 in E. coli, stain DH10B (Life Technologies, Gibco-BRL). Overnight cultures were grown in Luria Broth (LB) containing 100 mg/ml ampicillin. The next day, 500 ml of fresh LB was inoculated with 10 ml of overnight culture, and grown in a 2 liter flask at 37° C. with constant shaking until the culture reached an absorbance of 0.8 at 600 nm. Expression of the fusion protein was induced by addition of isopropyl b-D-thiogalactosidase (IPTG) to a final concentration of 0.05 mM. The cultures were shaken for three hours at room temperature, and the cells were harvested by centrifugation. The cell pellets were stored frozen until protein purification.

### Purification of P38 Kinase-Alpha

All chemicals were from Sigma Chemical Co. unless noted. Twenty grams of E. coli cell pellet collected from five 1 L shake flask fermentations was resuspended in a volume of PBS (140 mM NaCl, 2.7 mM KCl, 10 mM Na.sub.2 HPO.sub.4, 1.8 mM KH.sub.2 PO.sub.4, pH 7.3) up to 200 ml. The cell suspension was adjusted to 5 mM DTT with 2 M DTT and then split equally into five 50 ml Falcon conical tubes. The cells were sonnicated (Ultrasonics model W375) with a 1 cm probe for 3.times.1 minutes (pulsed) on ice. Lysed cell material was removed by centrifugation (12,000×g, 15 minutes) and the clarified supernatant applied to glutathione-sepharose resin (Pharmacia).

### Glutathione-Sepharose Affinity Chromatography

Twelve ml of a 50% glutathione sepharose —PBS suspension was added to 200 ml clarified supernatant and incubated batchwise for 30 minutes at room temperature. The resin was collected by centrifugation (600.times.g, 5 min) and washed with 2.times.150 ml PBS/1% Triton X-100, followed by 4.times.40 ml PBS. To cleave the p38 kinase from the GST-p38 fusion protein, the glutathione-sepharose resin was resuspended in 6 ml PBS containing 250 units thrombin protease (Pharmacia, specific activity >7500 units/mg) and mixed gently for 4 hours at room temperature. The glutathione-sepharose resin was removed by centrifugation (600.times.g, 5 min) and washed 2.times.6 ml with PBS. The PBS wash fractions and digest supernatant containing p38 kinase protein were pooled and adjusted to 0.3 mM PMSF.

### Mono Q Anion Exchange Chromatography

The thrombin-cleaved p38 kinase was further purified by FPLC-anion exchange chromatography. Thrombin-cleaved sample was diluted 2-fold with Buffer A (25 mM HEPES, pH 7.5, 25 mM beta-glycerophosphate, 2 mM DTT, 5% glycerol) and injected onto a Mono Q HR 10/10 (Pharmacia) anion exchange column equilibrated with Buffer A. The column was eluted with a 160 ml 0.1 M-0.6 M NaCl/Buffer A gradient (2 ml/minute flowrate). The p38 kinase peak eluting at 200 mM NaCl was collected and concentrated to 3-4 ml with a Filtron 10 concentrator (Filtron Corp.).

### Sephacryl S100 Gel Filtration Chromatography

The concentrated Mono Q-p38 kinase purified sample was purified by gel filtration chromatography (Pharmacia HiPrep 26160 Sephacryl S100 column equilibrated with Buffer B (50 mM HEPES, pH 7.5, 50 mM NaCl, 2 mM DTT, 5% glycerol)). Protein was eluted from the column with Buffer B at a 0.5 ml/minute flowrate and protein was detected by absorbance at 280 nm. Fractions containing p38 kinase (detected by SDS-polyacrylamide gel electrophoresis) were pooled and frozen at −80° C. Typical purified protein yields from 5 L E. coli shake flasks fermentations were 35 mg p38 kinase.

### Kinetics Assays

### Association Kinetics:

SKF-86002 (from Calbiochem; KD˜200 nM) gives an increase in fluorescence upon binding to p38a (as monitored by an excitation at 340 nm and emission at 420 nm). SKF-86002 (1-2 uM) was preincubated with p38a (20-60 nM) for 5-10 min at room temperature in a buffer consisting of 20 mM Bis-Tris, 2 mM EDTA, 500 mM NaCl, 0.01% NaN3, 0.15% NOG and 5% DMSO. The sample compound (20-100 nM) was then added and the change in fluorescence monitored. As SKF dissociated from its binding site on p38a, the SKF was replaced by the sample compound and a decrease in fluorescence was observed on a time scale proportional to the association rate of the compound. Using the known binding kinetics of SKF-86002, the association rate of the compound was measured.

### Dissociation Kinetics

Sample compounds (50 or 100 nM) were preincubated with p38a (37 nM protein or 21 nM as determined by active site titration) ove might at room temperature in a buffer consisting of 20 mM Bis-Tris, 2 mM EDTA, 0.01% NaN3, 0.15% NOG, 500 mM NaCl and 5% DMSO. The following day, SKF 86002 was added to a final concentration of 50 uM. The fluorescence increase observed up on the binding of SKF 86002 to p38a was monitored by excitation at 340 nm and emission at 420 nm, and the dissociation rate was measured.

### Solubility Assay:

Standard equilibrium solubility measurements are well known to the skilled person,

### Data:

In the present invention, the term “active”, “potent” or “potency” means that the compounds of formula (Ia) or formula (Ib) have an IC₅₀ (TNFα screen) of less than 200 nM as measured by the TNF assay described herein.

Preferably, the compounds of the invention have an IC₅₀ (TNFα screen) of less than 100 nM.

The examples were tested in the assay described above and were found to have an IC 53 (TNFα screen) of less than 10 nM:

| Example | TNF IC50 nM |
|---|---|
| 1 | 5.6 |
| 2 | 2.9 |
| 3 | 4.0 |
| 4 | 8.2 |
| 5 | 2.5 |
| 6 | 1.8 |
| 7 | 3.0 |
| 8 | 5.5 |
| 9 | 5.6 |
| 10 | 4.3 |
| 11 | 4.9 |

### Examples and Preparations

Nuclear magnetic resonance (NMR) data were obtained using Varian Unity Inova-400, Varian Unity Inova-300 or Bruker AC300 spectrometers and are quoted in parts per million from tetramethylsilane. Mass spectral (MS) data we re obtained on a Finnigan Mat. TSQ 7000 or a Fisons Instruments Trio 1000. The calculated and observed ions quoted refer to the isotopic composition of lowest mass. For column chromatography on silica gel, Kieselgel 60, 230-400 mesh, from E. Merck, Darmstadt was used, unless otherwise specified. Kieselgel 60 F₂₅₄ plates from E. Merck were used for TLC, and compounds were visualised using UV light, 5% aqueous potassium permanganate or Dragendorff's reagent (oversprayed with aqueous sodium nitrite). Water content was determined on a Mitsubishi CA100 (Coulometric Karl Fisher Titrator). Other measurements were taken using standard equipment.

PdCl₂(dppf).CH₂Cl₂ is 1,1-bis(diphenylphosphino)ferrocene palladium (II) chloride 1:1 dichloromethane complex.

DBU is 1,8-diazabicyclo[5.4.0]undec-7-ene.

LCMS conditions: Column 50×2 mm Luna II C18 5 μm, A is water+0.1% formic acid, B is acetonitrile+0.1% formic acid. Gradient:5% B 0.3 min to 95% B over 4.3 min, held at 95% B for 0.1 min then 5% B over 0.1 min. Flow rate: 1 mL/min.

### Preparation 1: 2-Chloro-4-hydrazinophenol hydrochloride

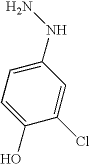

4-Amino-2-chlorophenol (25.0 g, 174 mmol) suspended in water (40 mL) and conc. hydrochloric acid (63 mL) at −10° C. under nitrogen. A solution of sodium nitrite (12.0 g, 174 mmol) in water (35 mL) was added dropwise and stirring continued at −10° C. for 30 minutes. Tin(II) chloride (98.1 g, 435 mmol) in hydrochloric acid (6M, 100 mL) was poured into the reaction and the temperature allowed to rise to 0° C. with stirring over 3.5 hours. The mixture was filtered and a wet white solid isolated.

### Preparation 2: 4-(5-Amino-3-tert-butylpyrazol-1-yl)-2-chlorophenol

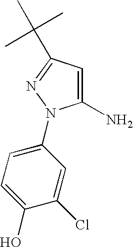

Prepared using a similar procedure used for the preparation of 7, using preparation 1 to yield a solid (65.79 g, >100%) containing the product.

### Preparation 3: 5-tert-Butyl-2-[4-(tert-butyldimethylsilanyloxy)-3-chlorophenyl]-2H-pyrazol-3-ylamine

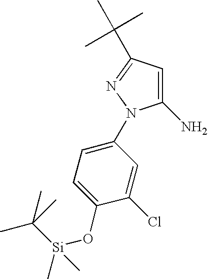

Preparation 2(40.0 g, 150 mmol) in N,N-dimethylformamide (100 mL) was treated with imidazole (11.2 g, 165 mmol) and tert-butyldimethylsilyl chloride (23.8.158 mmol) and stirred for 18 hours at RT. Methanol (40 mL) was added and the mixture concentrated, diluted with ethyl acetate (300 mL) and washed with sat. sodium hydrogencarbonate. The aqueous was diluted with water and re-extracted with ethyl acetate several times and the combined organics dried (Na₂SO⁻₄). The product was partially purified by chromatography (1:6 ethyl acetate in hexane and a little dichloromethane to aid solubility of product) and crystallised (ethyl acetate/hexane) to yield white needles (9.61 g, 17%).

### Preparation 4: {5-tert-Butyl-2-[4-(tert-butyldimethylsilanyloxy)-3-chlorophenyl]-2H-pyrazol-3-yl}carbamic acid phenyl ester

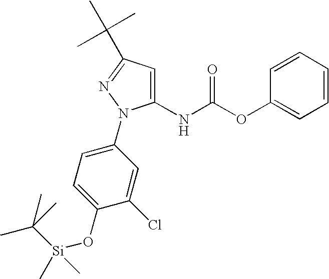

Phenyl chloroformate (1.81 g, 11.6 mmol) was added portion-wise to preparation 3 (4.0 g, 10.5 mmol) and pyridine (1.16 g, 14.7 mmol) in dry tetrahydrofuran (30 mL) at 0° C. The reaction mixture was allowed to warm to RT over 3 hours and the n diluted with ethyl acetate (100 mL), washed with water (100 mL) and dried (Na₂SO₄). After removal of the solvent the crude oil was triturated with heptane (=50 mL) and the white solid filtered off and dried in vacuo (4.5 g, 86%).

### Preparation 5: 5-Bromo-2-chloro-phenol

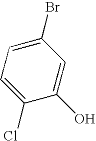

A solution of 5-bromo-2-chloroanisole (20 g, 90.3 mmol) in dichloromethane (100 ml) at 0° C. under nitrogen was treated with borontribromide (1M in dichloromethane, 100 mL, 0.1 mol) dropwise over 2.5 hours. After 10 minutes the reaction was allowed to warm to RT and stirred for 18 hours. The reaction mixture was poured into water (200 mL) and ice (200 mL) and stirred for 30 minutes, dichloromethane (100 mL) was added and the organics separated. The aqueous phase was saturated with sodium chloride and re-extracted with dichloromethane (2×200 mL). The combined organics were dried (MgSO₄) to furnish a white solid (18.34 g, 98%).

### Preparation 6: Di-tert-butyl 1-(4-chloro-3-hydroxyphenyl)hydrazine-1,2-dicarboxylate

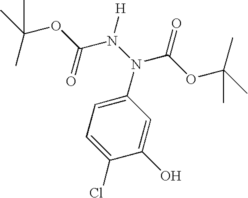

Preparation 5 (9.32 g, 44.9 mmol) in dry tetrahydrofuran (120 mL) at −78° C. under dry nitrogen was treated with n-butyllithium (2.5 M in hexanes, 45 mL, 112.3 mmol) dropwise over 30 minutes. After stirring at this temperature for 10 minutes di-tert-butyl azodicarboxylate (10.34 g, 44.9 mmol) was added in 3 portions over 30 minutes and the solution left to stir at −78° C. for 1 hour. The solution was then warmed to −10° C. and sat. ammonium chloride (100 mL) added. Ethyl acetate (150 mL) and water (150 mL) were added and organics separated, the aqueous was re-extracted with ethyl acetate (150 mL) and the combined organics dried (MgSO₄). The product was crystallised (ethyl acetate) to yield white crystals (5.24 g, 33%).

### Preparation 7: 5-(5-Amino-3-tert-butylpyrazol-1-yl)-2-chlorophenol

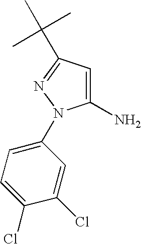

Preparation 6 (5.24 g, 14.6 mmol), pivavoylacetonitile (1.83 g, 14.6 mmol) and conc. hydrochloric acid (7 mL) in ethanol (50 mL) were heated to reflux under nitrogen for 3 hours. The reaction mixture was poured into water (50 mL) and neutralised with sat. sodium hydrogencarbonate (80 mL) and extracted with dichloromethane-methanol (9:1) (6×50 mL), the combined organics were dried (MgSO₄) and purified by:chromatography (6:1 dichloromethane:diethyl ether) to yield a green solid (2.55 g, 66%).

### Preparation 8: 5-tert-Butyl-2-[3-(tert-butyldimethylsilanyloxy)-4-chlorophenyl]-2H-pyrazol-3-ylamine

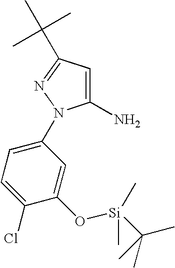

Preparation 7 (2.54 g, 9.6 mmol) in N,N-dimethylformamide (30 mL) was treated with imidazole (976 mg, 14.4 mmol) and tert-butyldimethylsilyl chloride (1.33 g, 9.6 mmol) and left to stir at RT for 16 h. The reaction mixture was diluted with ethyl acetate (75 mL) and washed with water (75 mL), brine (75 mL) and dried (MgSO₄). The product was purified by chromatography (1:1 pentane:dichloromethane) to give an orange solid (2.69 g, 74%).

### Preparation 9: {5-tert-Butyl-2-[3-(tert-butyldimethylsilanyloxy)-4-chlorophenyl]-2H-pyrazol-3-yl}carbamic acid phenyl ester

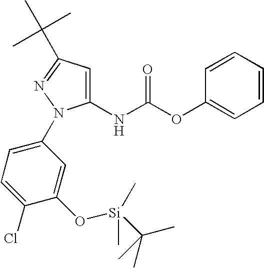

Prepared using the procedure for preparation 4, using preparation 8 to yield a white solid (6.38 g, 86%).

### Preparation 10: Di-tert-butyl 1-{3-[(dimethylamino)methyl]phenyl}hydrazine-1,2-dicarboxylate

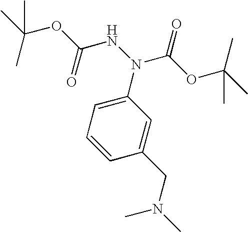

Prepared using the procedure for preparation 6, using (3-bromobenzyl)dimethylamine to yield a white solid (3.60 g, 99%).

### Preparation 11: 5-tert-Butyl-2-(3-dimethylaminomethylphenyl)-2H-pyrazol-3-ylamine

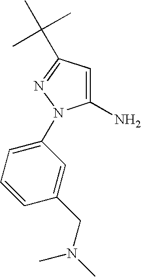

Prepared using the procedure for preparation 7, using preparation 10 to yield an orange oil (1.14 g, 43%).

### Preparation 12: (3-Hydrazinobenzyl)dimethylamine

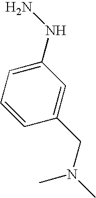

Preparation 10 (1.98 g, 5.0 mmol) in dichloromethane (20 mL) and methanol (10 mL) was cooled to 0° C. and hydrogen chloride (g) was bubbled through for 20 minutes. Stirring was continued at 0° C. for 2 hours and at RT for 20 hours. Hydrogen chloride (g) was bubbled through again at RT and the mixture left to stir at RT for 3 hours. The solvents were removed and re-evaporated from ethanol and methanol to leave a brown foam (1.38 g, 91%).

### Preparation 13: 2,2-Dimethyl-3-methylsulfanyl-propionic acid methyl ester

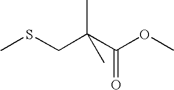

N,N-Diisopropylethylamine (15.5 g, 0.12 mol) was added to a solution of methyl 2,2-dimethyl-3-hydroxypropionate (13.2 g, 0.1 mol) in dichloromethane (150 mL) and the solution was cooled to 0° C. Methanesulfonyl chloride (12.6 g, 0.11 mol) was then added dropwise and the mixture was stirred at 0° C. for 90 minutes. The reaction mixture was then diluted with 0.5 M hydrochloric acid (100 mL) and the layers were separated. The aqueous was extracted with dichloromethane (2×50 mL) and the combined organic solution was dried (MgSO₄) and concentrated in vacuo. Methanethiol sodium salt (7.7 g, 0.11 mol) was added to a solution of the residue in dioxan (100 mL) and the mixture was heated under reflux for 24 hours. The mixture was then diluted with ethyl acetate (250 mL), washed with water and brine, dried (MgSO₄) and concentrated in vacuo. The product was purified by column chromatography (50-100% dichloromethane in pentane) to afforded the compound as a pale yellow oil (3.85 g, 24%).

### Preparation 14: 4,4-Dimethyl 5-methylsulfanyl-3-oxo-pentanenitrile

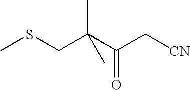

A suspension of sodium hydride (60% dispersion in mineral oil, 1.20 g, 30 mmol) in tetrahydrofuran (20 mL) was brought to reflux. A solution of preparation 13 (3.84 g, 23.7 mmol) in acetonitrile (1.56 mL, 30 mmol) was added and the mixture was heated under reflux for 3 hours. The cooled reaction mixture was then diluted with water, acidified with 2M hydrochloric acid (30 mL) and extracted with dichloromethane (3×50 mL). The combined organic extracts were dried (MgSO₄), concentrated in vacuo and the residue was purified by chromatography (dichloromethane) to afford the compound as a pale yellow oil (2.70 g, 67%).

### Preparation 15: 2-(3-Dimethylaminomethylphenyl)-5-(1,1-dimethyl-2-methylsulfanylethyl)-2H-pyrazol-3-ylamine

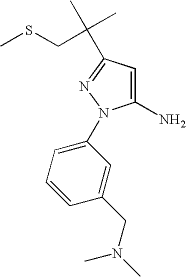

Prepared using the procedure for preparation 7, using preparation 14 and preparation 12 to yield an orange oil (417 mg, 53%).

### Preparation 16: 4-(3-Bromobenzyl)morpholine

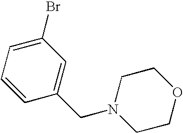

1-Bromo-3-bromomethylbenzene (16 g, 64 mmol) in dry tetrahydrofuran (30 mL) was treated with morpholine (19.5 g, 224 mmol) and the resulting solution stirred at RT for 17 hours. Ethyl acetate (250 mL) was added and washed with water (150 mL). The aqueous layer was basified to pH 14 with 5M sodium hydroxide and saturated with sodium chloride and re-extracted with the organic layer, followed by extraction with ethyl acetate (2×100 mL). The combined organic extracts were dried (MgSO₄) leaving a yellow oil (16.4 g, 100

### Preparation 17: Di-tert-butyl 1-[3-(morpholin-4-ylmethyl)phenyl]hydrazine-1,2-dicarboxylate

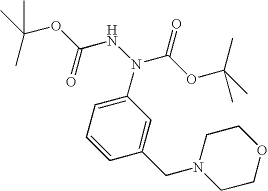

Prepared using the procedure for preparation 6, using preparation 16 to yield an orange oil (8.25 g, 52%).

### Preparation 18: (3-Morpholin-4-ylmethylphenyl)hydrazine trihydrochloride

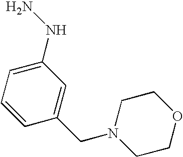

Preparation 17 (4.07 g, 10 mmol) in dichloromethane (50 mL) and methanol (50 mL) was cooled to 4° C. and saturated with hydrogen chloride gas. The mixture was stirred at 4° C. for 3 hours and at RT for 17 hours. The solvents were removed and the material was a zeotroped with methanol (×3) before being triturated with diethyl ether to leave a yellow solid (2.96 g, 93%)

### Preparation 19: 5-tert-Butyl-2-(3-morpholin-4-ylmethylphenyl)-2H-pyrazol-3-ylamine

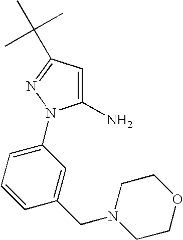

Prepared using the procedure for preparation 7, using preparation 18 to yield white solid (1.65 g, 58%).

### Preparation 20: [2-(3-Bromophenoxy)ethyl]dimethylamine

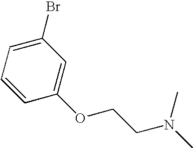

3-Bromophenol (4.80, 30.6 mmol), chloroethyldimethylamine hydrochloride (8.64 g, 60 mmol) and anhydrous potassium carbonate (16.56 g, 120 mmol) in N,N-dimethylformamide (40 mL) were stirred at RT for 2 hours. Potassium iodide (332 mg, 2.0 mmol) was added and stirring continued for 24 hours at RT then at 40° C. for 24 hours. Further aliquots of chloroethyldimethylamine hydrochloride (8.64 g, 60 mmol) and potassium carbonate (8.28 g, 60 mmol) and water (100 mL) were added and the mixture heated to 60° C. for 4 hours, water (20 mL) was added and heating continued for 20 hours. The cooled reaction was diluted with ethyl acetate, washed with water, 0.5 M sodium hydroxide, brine and dried (MgSO₄). The product was purified by chromatography (ethyl acetate) to leave a yellow oil (1.76 g, 24%).

### Preparation 21: Di-tert-butyl 1-(3-[2-(dimethylamino)ethoxy]phenyl)hydrazine-1,2-dicarboxylate

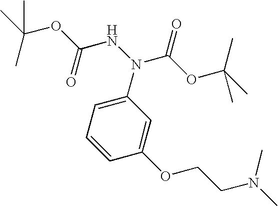

Prepared using the procedure for preparation 6, using preparation 20 to yield a brown foam (1.38 g, 91%).

### Preparation 22: 5-tert-Butyl-2-[3-(2-dimethylaminoethoxy)phenyl]-2H-pyrazol-3-ylamine

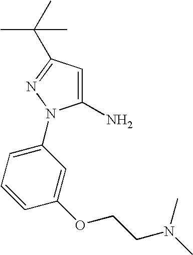

Prepared using the procedure for preparation 7, using preparation 21 to yield an orange oil (555 mg, 81%).

### Preparation 23: (5-Bromopyridin-2-yl)hydrazine

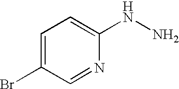

2-Chloro-5-bromopyridine (64 g, 333 mmol) was suspended in hydrazine monohydrate (250 mL) and the mixture was heated at 70° C. for 72 hours. The reaction mixture was then diluted with water (750 mL) and the resulting precipitate was filtered off and azeotroped, firstly with toluene (×2) then dichloromethane (×2), to afford the title compound as a pale brown solid (52 g, 83%).

### Preparation 24: N-(5-Bromopyridin-2-yl)-N′-(4-diethoxymethylbenzylidene)hydrazine

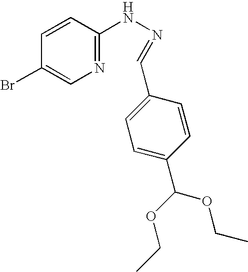

4-Diethoxymethylbenzaldehyde (5.0 g, 24.04 mmol) and preparation 23 (4.47 g, 24.04 mmol) were heated in ethanol (75 ml) to reflux for 3 hours. The mixture was allowed to cool and the product filtered off, washed with ethanol and dried to give a yellow solid (7.92 g). NMR analysis shows product and analogous (unprotected) aldehyde (1:1).

### Preparation 25: 6-Bromo-3-(4-diethoxymethylphenyl)[1,2,4]triazolo[4,3-a]pyridine

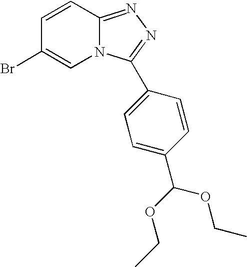

Iodobenzene diacetate (9.29 g, 28.8 mmol) was slowly added to preparation 24 (7.92 g) in dichloromethane (75 mL) and ethanol (10 mL) and the mixture stirred at RT for 20 h. The solution was diluted with dichloromethane (50 mL), washed with 1M NaOH (100 mL), brine (100 mL) and dried (MgSO₄). Removal of the solvent left an orange/yellow solid, which was purified by chromatography (0-100% ethyl acetate in heptane) to yield a fawn solid (4.39 g, 98% of anticipated yield) as the title compound and another fawn solid as 4-(6-bromo-[1,2,4]triazolo[4,3-a]pyridin-3-yl)benzaldehyde (2.21 g, 66% of anticipated yield)

### Preparation 26: 6-Bromo-3-(4-morpholin-4-ylmethylphenyl)-[1,2,4]triazolo[4,3-a]pyridine

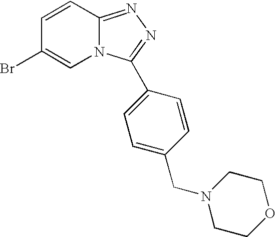

The aldehyde from preparation 25 (1.10 g, 3.60 mmol) and morpholine (635 mg, 7.3 mmol) were stirred in dichloromethane (20 mL) for 30 minutes. Sodium triacetoxyborohydride (1.54 g, 7.3 mmol) was added and the reaction stirred at RT for 18 hours, followed by a further aliquot of sodium triacetoxyborohydride (1.15 g, 5.4 mmol) and stirring for 24 hours. Dichloromethane (40 mL) was added and the organics washed with sodium hydroxide (1M, 40 mL), brine (2×20 mL) and dried (Na₂SO₄). The product was purified by chromatography (0-10% methanol in dichloromethane+1% ammonia) to yield a yellow solid (1.2 g, 89%).

### Preparation 27: {2-[3-(4-Morpholin-4-ylmethylphenyl)-[1,2,4]triazolo[4,3-a]pyridin-6-ylsulfanyl]phenyl)}methanol

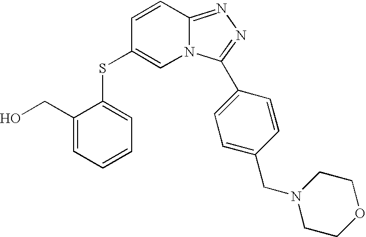

A solution of preparation 26 (1.20 g, 3.20 mmol) in N,N-dimethylformamide was degassed with Ar for 30 minutes. 2-Mercaptobenzylalcohol (631 g, 4.50 mmol), caesium carbonate (21.0 g, 6.4 mmol) and 1,1′-bis(diphenylphosphino)ferrocenedichloropalladium(II) dichloromethane adduct (525 mg, 0.6 mmol) were added and the mixture degassed for a further 10 minutes then heated to 90° C. for 2.5 hours. The mixture was diluted with ethyl acetate (40 mL), washed with water (40 mL), methanol (8 mL) was added to the organics which were then washed with brine (2×30 mL) and dried (Na₂SO₄). The material was purified by chromatography (0-10% methanol in dichloromethane+1% ammonia) to yield a brown oil (920 mg, 67%).

### Preparation 28: 2-[3-(4-Morpholin-4-ylmethylphenyl)-[1,2,4]triazolo[4,3-a]pyridin-6-ylsulfanyl]-benzylamine

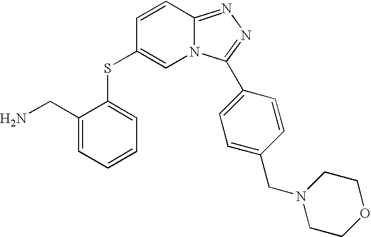

Preparation 27 (920 mg, 2.1 mmol) and N,N-diisopropylethylamine (431 mg, 4.3 mmol) in dichloromethane (20 mL) were treated with methanesulfonic anhydride (556 mg, 3.2 mmol) and the reaction allowed to stir at RT 2.5 hours. The reaction mixture was poured into methanolic ammonia (7M, 30 ml) and stirred at RT for 18 h. The solvents were removed and the resulting mixture diluted with dichloromethane (30 mL), washed with sat. sodium hydrogencarbonate (30 mL), brine (30 ml) and dried (Na₂SO₄). The product was purified by chromatography (0-10% methanol in dichloromethane+1% ammonia) to yield a brown oil (340 mg, 37%).

### Preparation 29: 5-Benzyloxy-2-chlorobenzoic acid benzyl ester

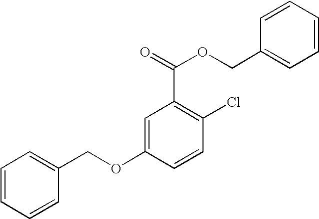

2-Chloro-5-hydroxybenzoic acid (1.0 g, 5.79 mmol), benzyl bromide (2.0 g, 11.70) and potassium carbonate (3.0 g, 21.47 mmol) in acetonitrile were heated to reflux for 3 hours. The reaction was filtered, solvent removed, diluted with diethyl ether, washed with 1 M HCl and dried (Na₂SO₄) to leave a clear oil (2.3 g including some excess benzyl bromide).

### Preparation 30: [5-(Benzyloxy)-2-chlorophenyl]methanol

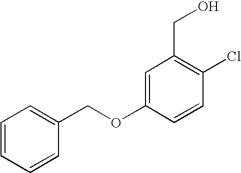

Preparation 29 (2.3 g) was dissolved in dry tetrahydrofuran (100 mL) and lithium aluminiumhydride (1M in tetrahydrofuran 8 mL, 8.0 mmol) was added slowly at RT. After 1 hour the reaction was quenched by the addition of water (1 mL), sodium hydroxide (1M, 1 mL), and dried (Na₂SO₄) to leave a clear oil. The product was obtained by distillation (100° C., 0.05 mbar) (1.12 g, 78%).

### Preparation 31: 5-(Benzyloxy)-2-chlorobenzaldehyde

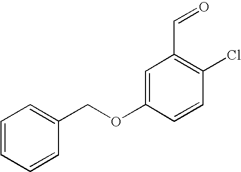

Preparation 30 (18.5 g, 74.4 mmol) in dichloromethane was treated with manganese dioxide (31.4 g, 361 mmol) and left to stir for 72 h. The mixture was filtered and the solvent evaporated leaving an oil which crystallised on standing. The product was recrystallised (diisopropylether, 40 mL) (12.3 g, 50%).

### Preparation 32: 3-[5-(Benzyloxy)-2-chlorophenyl]-6-bromo[1,2,4]triazolo[4,3-a]pyridine

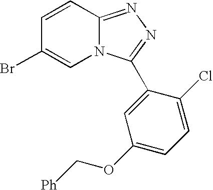

Preparation 31 (9.98 g, 41.07 mmol) and preparation 23 (7.72 g, 41.07 mmol) in a mixture of dichloromethane (100 mL) and methanol (20 mL) were heated for 2 hours. The reaction was cooled and the solids filtered off. The solids were suspended in a mixture of dichloromethane (100 mL) and methanol (20 mL) and iodobenzene diacetate (13.2 g, 41.0 mmol) added and the mixture stirred at RT for 2 hours then the solvents were removed. The product was triturated from diethyl ether (10.21 g, 60%)

### Preparation 33: {2-[3-(5-Benzyloxy-2-chlorophenyl)-[1,2,4]triazolo[4,3-a]pyridin-6-ylsulfanyl]phenyl}methanol

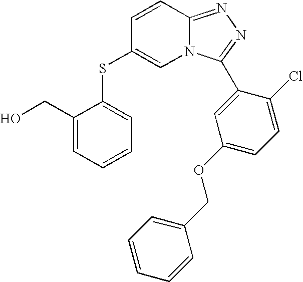

Prepared using the procedure for preparation 27, using preparation 32 to yield crystals (9.0 g, 71%).

### Preparation 34: 6-{[2-(Azidomethyl)phenyl]thio}-3-[5-(benzyloxy)-2-chlorophenyl][1,2,4]triazolo[4,3-a]pyridine

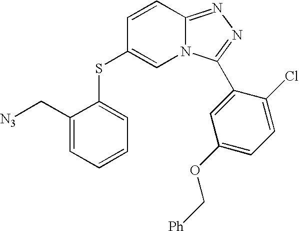

Preparation 33 (9.0 g, 18.94 mmol) in tetrahydrofuran was treated with diphenylphosphorylazide (5.0 ml, 22.79 mmol) and DBU (3.43 ml, 22.79 mmol) and left to stir for 16 hours. The solvent was removed and the crude material was dissolved in dichloromethane (600 mL), washed with water (2×100 mL) and the solvent removed. The product was purified by chromatography (2-5% methanol in dichloromethane) to leave an oil (9.3 g, 91%).

### Preparation 35: [2-((3-[5-(Benzyloxy)-2-chlorophenyl][1,2,4]triazolo[4,3-a]pyridin-6-yli)thio)benzyl]amine hydrochloride

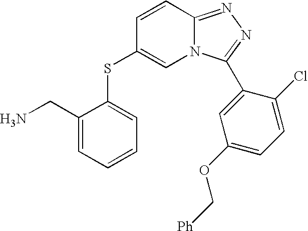

Preparation 34 (6.2 g, 12.4 mmol) in tetrahydrofuran was treated with water (0.27 mL, 14.9 mmol) and triphenylphosphine (3.92 g, 14.2 mmol) and stirred for 16 hours at RT. The solvent was removed and the product taken up in dichloromethane (100 mL), HCl (4M in dioxane, 9 ml, 36.0 mmol) was added and the solution stirred for 18 hours. Water was added (1 mL) and stirring continued for 72 hours. The mixture was filtered off and dried (3.61 g, 61%).

### Preparation 36: 3-[6-(2-Aminomethylphenylsulfanyl)-[1,2,4]triazolo[4,3-a]pyridin-3-yl]-4-chlorophenol hydrobromide

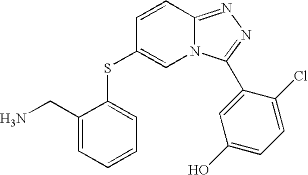

Prepared using the procedure for preparation 41, using preparation 35 yielding a solid (8.8 g, >100%).

### Preparation 37: N-(2-Benzyloxy-5-chlorobenzylidene)-N′-(5-bromopyridin-2-yl)hydrazine

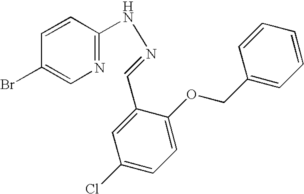

Prepared using the procedure for preparation 24, using preparation 23 and 2-benzyloxy-5-chlorobenzaldehyde yielding a white solid (94%).

### Preparation 38: 3-[2-(Benzyloxy)-5-chlorophen yl]-6-bromo[1,2,4]triazolo[4,3-a]pyridine

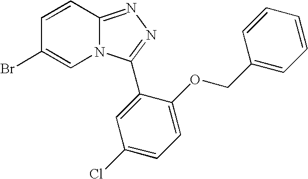

Prepared using the procedure for preparation 25, using preparation 37, the compound being triturated with diethyl ether (5.7 g, 92%).

### Preparation 39: {2-[3-(2-Benzyloxy-5-chloro-phenyl)-[1,2,4]triazolo[4,3-a]pyridin-6-ylsulfanyl]phenyl}methanol

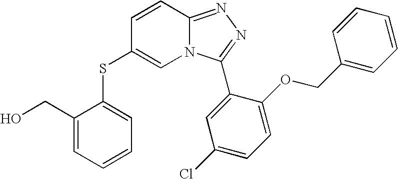

Prepared using the procedure for preparation 27, using preparation 38 (37%).

### Preparation 40: [2-({3-[2-(Benzyloxy)-5-chlorophenyl][1,2,4]triazolo[4,3-a]pyridin-6-yl}thio)benzyl]amine

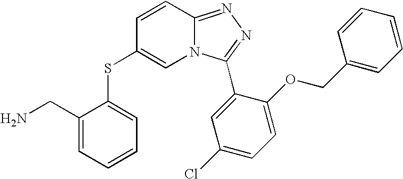

Prepared using a similar procedure used for the preparation of 28, using preparation 39 to yield a pale brown gum (52%).

### Preparation 41: 2-(6-{[2-(Aminomethyl)phenyl]thio}[1,2,4]triazolo[4,3-a]pyridin-3-yl)-4-chlorophenol hydrobromide

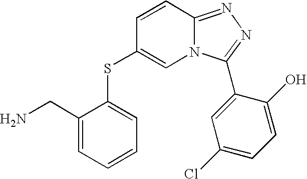

Preparation 40 (16.3 g, 34.5 mmol) in hydrogen bromide in acetic acid (35 mL) was stirred for 18 hours at RT. Diethyl ether (250 ml) was added and stirred and the solvent decanted off, more diethyl ether (300 mL) was added and the mixture stirred for 5 hours and the precipitate was filtered and azeotroped with toluene, ethanol and diethyl ether before being dried (P 205) (15.6 g, 95%).

Preparation 42: N-(3-Benzyloxy-benzylidene)-N′-(5-bromopyridin-2-yl)hydrazine

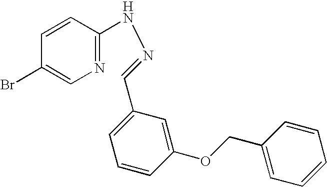

Prepared using the procedure for preparation 24, using preparation 23 and 3-benzyloxybenzaldehyde to yield a pale pink solid (10.17 g, 89%).

### Preparation 43: 3-(3-Benzyloxy-phenyl)-6-bromo[1,2,4]triazolo[4,3-a]pyridine

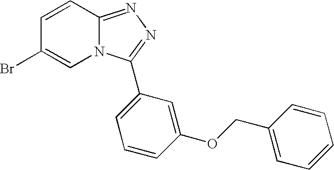

Prepared using the procedure for preparation 25, using preparation 42 to yield crystals (9.1 g, 89%).

### Preparation 44: {2-[3-(3-Benzyloxyphenyl)-[1,2,4]triazolo[4,3-a]pyridin-6-ylsulfanyl]phenyl}methanol

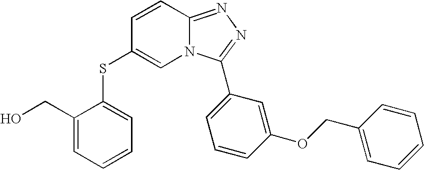

Prepared using the procedure for preparation 27, using preparation 43 to yield a brown solid (7.35 g, 71%).

### Preparation 45: 2-[3-(3-Benzyloxyphenyl)-[1,2,4]triazolo[4,3-a]pyridin-6-ylsulfanyl]benzylamine

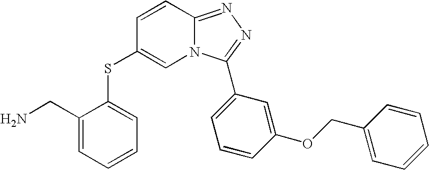

Prepared using the procedure for preparation 28, using preparation 44 to yield a brown foam (4.87 g, 67%).

### Preparation 46: 3-[6-(2-Aminomethylphenylsulfanyl)-[1,2,4]triazolo[4,3-a]pyridin-3-yl]phenol

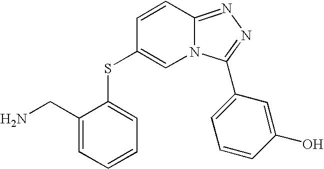

Borontribromide (1M in dichloromethane, 54 mL, 54 mmol) was added to a solution of preparation 45 (4.75 g, 10.8 mmol) at −70° C. for 10 minutes, then allowed to warm to 0° C. The mixture was re-cooled to −70° C. and methanol (20 mL) added followed by sat. sodium hydrogencarbonate solution until neutral pH. The product was extracted with dichloromethane-methanol (85:15, 5×50 mL) and dried (Na₂SO₄) leaving a dark oil which was triturated with ethyl acetate to leave a granular brown solid. The material was purified by chromatography (0-20% methanol in dichloromethane+1% ammonia) to leave a brown solid after ethyl acetate trituration (580 mg, 15%).

### Preparation 47: {2-[3-(3-Hydroxy-phenyl)-[1,2,4]triazolo[4,3-a]pyridin-6-ylsulfanyl]benzyl}carbamic acid tert-butyl ester

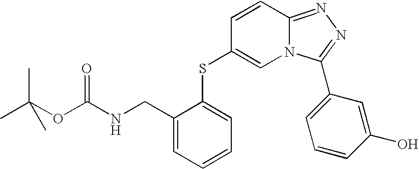

Preparation 46 (1.28 g, 3.68 mmol), di-tert-butyl dicarbonate (2.00 g, 9.20 mmol) and N,N-diisopropylethylamine (1.42 g, 11.03 mmol) were stirred in N,N-dimethylformamide (25 mL) at RT for 90 minutes. The solvent was removed and the mixture diluted with tetrahydrofuran (50 mL), water (50 mL) and lithium hydroxide hydrate (773 mg, 18.4 mmol) added and the mixture stirred at RT for 20 h. Diluted with ethyl acetate (25 mL), acidified with 1M citric acid, separated, organics washed with brine (30 mL) and dried (MgSO₄). Dioxane (50 mL), water (50 ml) and sodium carbonate (3.90 g, 36.8 mmol) were added and the mixture heated to 70° C. for 20 h. Acidified with citric acid, extracted with ethyl acetate (50 mL), washed with brine (50 mL) and dried (MgSO₄) to leave a fawn foam.

### Preparation 48: (2-{3-[3-(2-Morpholin-4-yl-ethoxy)phenyl]-[1,2,4]triazolo[4,3-a]pyridin-6-ylsulfanyl}benzyl)carbamic acid tert-butyl ester

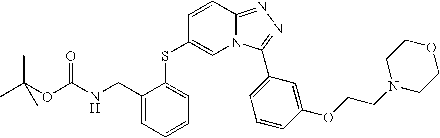

Preparation 47 (662 mg, 1.48 mmol), N-(2-chloroethyl)morpholine hydrochloride (330 mg, 1.77 mmol) and anhydrous potassium carbonate (612 mg, 4.43 mmol) in N,N-dimethylformamide (10 mL) were heated to 60° C. for 6 hours. The mixture was diluted with ethyl acetate (40 ml) and washed with brine (2×50 mL) and dried (MgSO₄) to leave a brown gum (900 mg).

### Preparation 49: 2-{3-[3-(2-Morpholin-4-yl-ethoxy)phenyl]-[1,2,4]triazolo[4,3-a]pyridin-6-ylsulfanyl}benzylamine

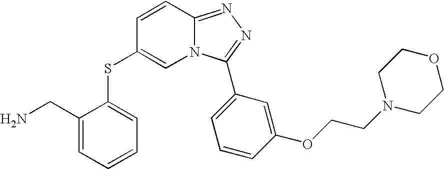

Preparation 48 (900 mg) in dichloromethane (5 mL) was treated with trifluoroacetic acid (1 mL), and the resulting mixture stirred at RT for 20 hours. The mixture was diluted with dichloromethane (20 ml), washed with 1M sodium hydroxide (30 mL), brine (30 mL) and dried (MgSO₄). The crude material was purified by chromatography (0-4% methanol in dichloromethane+1% ammonia) to yield a brown gum (475 mg, 69%).

### Preparation 50:1-{5-tert-Butyl-2-[4-(tert-butyldimethylsilanyloxy)-3-chloro-phenyl]-2H-pyrazol-3-yl}-3-(2-{3-[3-(2-morpholin-4-yl-ethoxy)phenyl]-[1,2,4]triazolo[4,3-a]pyridin-6-ylsulfanyl}benzyl)urea

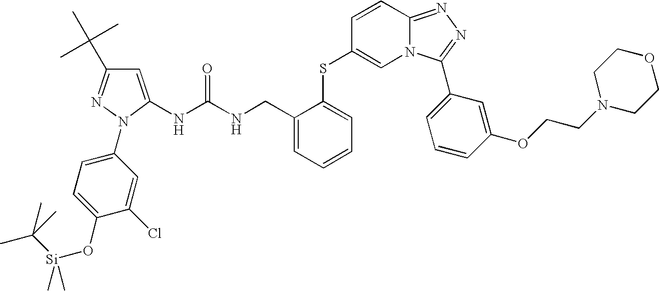

Preparation 49 (235 mg, 510 μmol) and preparation 4 (255 mg, 510 mmol) in dimethylsulfoxide (5 mL) were stirred at RT for 20 h. The mixture was diluted with ethyl acetate (50 mL), washed with brine (2×30 ml) and dried (MgSO₄). The crude material was purified by chromatography (0-5% methanol in dichloromethane+1% ammonia) to yield a fawn foam (314 mg, 71%).

### Preparation 61: 1-{5-tert-Butyl-2-[3-(tert-butyldimethylsilanyloxy)-4-chloro-phenyl]-2H-pyrazol-3-yl}-3-(2-{3-[3-(2-morpholin-4-yl-ethoxy)phenyl]-[1,2,4]triazolo[4,3-a]pyridin-6-ylsulfanyl}benzyl)urea

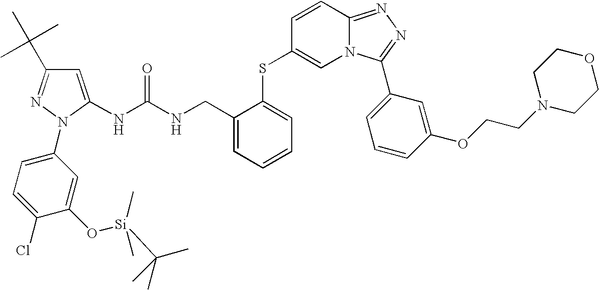

Prepared using the procedure for preparation 50, using preparation 49 and preparation 9 to yield a fawn foam (355 mg, 80%).

### EXAMPLES

### Example 1

### 1-{5-tert-Butyl-2-[3-(2-dimethylaminoethoxy)phenyl]-2H-pyrazol-3-yl}-3-{2-[3-(5-chloro-2-hydroxyphenyl)-[1,2,4]triazolo[4,3-a]pyridin-6-ylsulfanyl]benzyl}urea

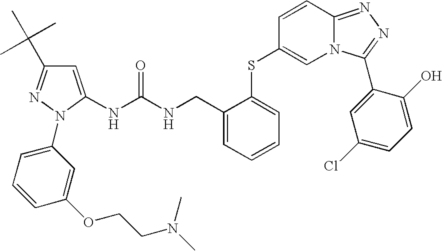

Preparation 22 (550 mg, 1.82 mmol) in dry tetrahydrofuran (16 mL) was treated with phenyl chloroformate (313 mg, 2.0 mmol) drop-wise at RT. After stirring for 1 hour a quarter of the material was mixed with preparation 41 (231 mg, 460 μmol), and N,N-diisopropylethylamine (259 mg, 2.0 mmol) in dimethylsulfoxide (5 mL) and the mixture heated to 60° C. for 1 hour, then at RT for 72 hours. Ethyl acetate was added and the organics washed with water and brine and dried (MgSO₄). The product was purified by chromatography (0-15% methanol in ethyl acetate and 1% ammonia) and crystallised (acetone) (126 mg, 39%).

¹H NMR (300 MHz, DMSO_{d6}) δ: 1.24 (9H, s), 2.18 (6H, s), 2.60 (2H, t), 4.06 (2H, t), 4.37 (2H, d), 6.24 (1H, s), 6.89-6.94 (1H, m), 6.98-7.07 (4H, m), 7.20 (3H, m), 7.17-7.30 (2H, m), 7.35 (1H, t), 7.44 (1H, dd), 7.54 (1H, d), 7.83 (1H, dd), 8.10 (1H, s), 8.31 (1H, s).

LRMS: m/z ES/APCI-711/713 [MH]⁺, 709/711 [M-H]⁻

### Example 2

### 1-{2-[3-(2-Chloro-5-hydroxy-phenyl)-[1,2,4]triazolo[4,3-a]pyridin-6-ylsulfanyl]benzyl}-3-[2-(3-dimethylaminomethyl-phenyl)-5-(1,1-dimethyl-2-methylsulfanylethyl)-2H-pyrazol-3-yl]urea

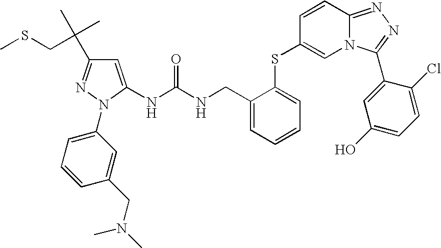

Prepared using the procedure for example 1, using preparation 36 and preparation 15 to yield a white solid (46 mg, 6%)

¹H NMR (300 MHz, DMSO_{d6}) δ: 1.29 (6H, s), 1.97 (3H, s), 2.14 (6H, s), 2.77 (2H, s), 3.42 (2H, s), 4.36 (2H, d), 6.26 (1H, s), 6.91 (1H, t), 6.97-7.04 (1H, m), 7.08-7.12 (1H, m), 7.17-7.49 (10H, m), 7.84 (1H, dd), 8.14-8.17 (1H, m), 8.27 (1H, s), 9.82 (1H, s).

LRMS: m/z APCI/ES 727/729 [MH]⁺, 727/725 [M-H]⁻

### Example 3

### 1-[5-tert-Butyl-2-(3-dimethylaminomethylphenyl)-2H-pyrazol-3-yl]-3-(2-[3-(5-chloro-2-hydroxyphenyl)-[1,2,4]triazolo[4,3-a]pyridin-6-ylsulfanyl]benzyl)urea

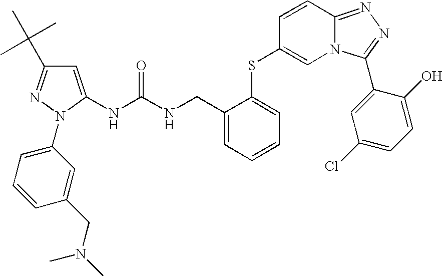

Prepared using the procedure for example 1, using preparation 11 and preparation 41 to yield a white solid (187 mg, 29%).

¹H NMR (300 MHz, DMSO_{d6}) δ: 1.24 (9H, s), 2.13 (6H, s), 3.39 (2H, s), 4.36 (2H, d), 6.23 (1H, s), 6.94-7.06 (2H, m), 7.18-7.30 (7H, m), 7.31-7.50 (4H, m), 7.53 (1H, d), 7.82 (1H, d), 8.08 (1H, s), 8.24-8.35 (1H, bs).

LRMS: m/z APCI/ES 681/679 [M-H]⁻.

### Example 4

### 1-[5-tert-Butyl-2-(3-dimethylaminomethylphenyl)-2H-pyrazol-3-yl]-3-{2-[3-(2-chloro-5-hydroxy-phenyl)-[1,2,4]triazolo[4,3-a]pyridin-6-ylsulfanyl]benzyl}urea

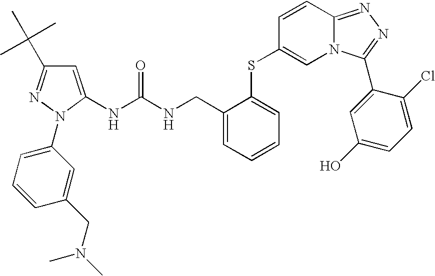

Prepared using the procedure for example 1, using preparation 11 and preparation 36 to yield a white solid (134 mg, 21%).

¹H NMR (300 MHz, DMSO_{d6}) δ: 1.24 (9H, s), 2.13 (6H, s), 3.40 (2H, s), 4.36 (2H, d), 6.23 (1H, s), 6.94-7.01 (1H, bs), 7.02 (1H, d), 7.18-7.29 (7H, m), 7.31-7.43 (3H, m), 7.44 (1H, dd), 7.53 (1H, d), 7.82 (1H, dd), 8.08 (1H, s), 8.28-8.35 (1H, bs).

LCMS: m/z ES 679 [M-H]⁻.

### Example 5

### 1-[5-tert-Butyl-2-(3-morpholin-4-ylmethylphenyl)-2H-pyrazol-3-yl]-3-{2-[3-(5-chloro-2-hydroxyphenyl)-[1,2,4]triazolo[4,3-a]pyridin-6-ylsulfanyl]benzyl}urea

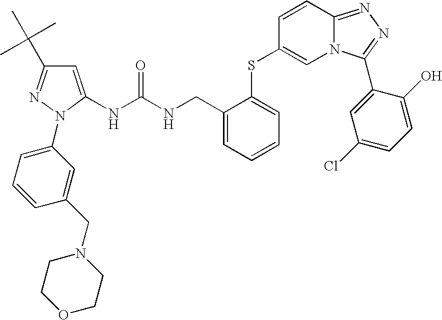

Preparation 19 (315 mg, 1.0 mmol) in dry tetrahydrofuran (3 mL) was cooled (ice-water) and treated with phenyl chloroformate (125 μL, 1.0 mmol), after stirring for 1 hour half of the mixture was added to a solution of preparation 41 (208 mg, 380 μmol) and N,N-diisopropylethylamine (66 μL, 380 μmol) in dimethylsulfoxide (2 mL) and left to stir at RT for two weeks. Ethyl acetate was added (30 mL) and the organics washed with water (2×15 mL), brine and dried (MgSO₄). The product was purified by chromatography (O-20% methanol in ethyl acetate+2% ammonia) (7.5 mg, 4%).

¹H NMR (300 MHz, CD 3OD) δ: 1.31 (9H, s), 2.39-2.46 (4H, m), 3.53 (2H, s), 3.59-3.64 (4H, m), 4.48 (2H, s), 6.28 (1H, s), 6.96 (1H, d), 7.26-7.45 (10H, m), 7.54 (1H, d), 7.71 (1H, d), 7.80-7.82 (1H, bs)

LRMS: m/z APCI/ES 723/725 [MH]⁺.

### Example 6

### 1-[5-tert-Butyl-2-(3-morpholin-4-ylmethylphenyl)-2H-pyrazol-3-yl]-3-{2-[3-(2-chloro-5-hydroxyphenyl)-[1,2,4]triazolo[4,3-a]pyridin-6-ylsulfanyl]benzyl}urea

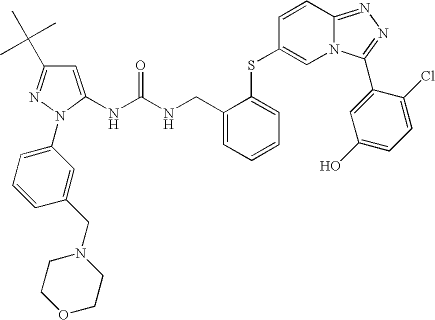

Prepared using the procedure for example 5, using preparation 19 and preparation 36 (7.6 mg, 4%).

¹H NMR (300 MHz, CD₃OD) δ: 1.31 (9H, s), 2.38-2.49 (4H, m), 3.54 (2H, s), 3.58-3.65 (4H, m), 4.45 (2H, s), 6.27 (1H, s), 7.00-7.06 (2H, m), 7.26-7.45 (10H, m), 7.69-7.71 (1H, bs), 7.74 (1H, d).

LRMS: m/z APCI/ES 723/725 [MH]⁺.

### Example 7

### 1-[5-tert-Butyl-2-(4-chloro-3-hydroxyphenyl)-2H-pyrazol-3-yl]-3-{2-[3-(4-morpholin-4-ylmethylphenyl)-[1,2,4]triazolo[4,3-a]pyridin-6-ylsulfanyl]benzyl}urea

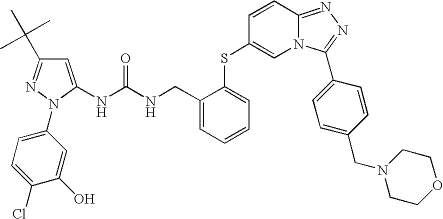

Preparation 28 (113 mg, 0.3 mmol) and preparation 9 (131 mg, 0.3 mmol) in dimethyl sulfoxide (2 ml) were stirred at RT for 20 hours. Triethylamine trihydrofluoride (42 μL, 0.3 mmol) was added and the reaction stirred at RT for 20 hours. Ethyl acetate (20 mL) was added and the organics washed with water (30 mL) and dried (Na₂SO₄). The product was purified by chromatography (0-10% methanol in dichloromethane+1% ammonia) to yield a white solid (50 mg, 23%).

¹H NMR (300 MHz, CD₃OD) δ: 1.25 (9H, s), 2.47-2.52 (4H, bs), 3.62 (2H, s), 3.69-3.74 (4H, m), 4.50 (2H, s), 6.18 (1H, s), 6.84 (1H, dd), 7.01 (1H, d), 7.25 (1H, dd), 7.28-7.42 (4H, m), 7.47 (1H, dd), 7.55 (2H, d), 7.68 (1H, dd), 7.74 (2H, d), 8.06-8.09 (1H, m).

LCMS: m/z ES 721 [M-H]⁻, RT=2.47 min.

### Example 8

### 1-[5-tert-Butyl-2-(3-chloro-4-hydroxyphenyl)-2H-pyrazol-3-yl]-3-{2-[3-(4-morpholin-4-ylmethylphenyl)-[1,2,4]triazolo[4,3-a]pyridin-6-ylsulfanyl]benzyl}urea

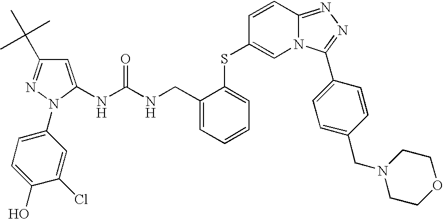

Prepared using the procedure for example 7, using preparation 28 and preparation 4 to yield a white solid (20 mg, 9%).

¹H NMR (300 MHz, CD 3OD) δ: 1.25 (9H, s), 2.47-2.52 (4H, bs), 3.62 (2H, s), 3.69-3.74 (4H, m), 4.50 (2H, s), 6.16 (1H, s), 6.96 (1H, d), 7.14 (1H, dd), 7.25 (1H, dd), 7.28-7.42 (4H, m), 7.47 (1H, dd), 7.55 (2H, d), 7.68 (1H, dd), 7.74 (2H, d), 8.06-8.08 (1H, m).

LCMS: m/z ES 721 [M-H]⁻, RT=2.63 min.

### Example 9

### 1-[5-tert-Butyl-2-(3-chloro-4-hydroxyphenyl)-2H-pyrazol-3-yl]-3-(2-{3-[3-(2-morpholin-4-ylethoxy)phenyl]-[1,2,4]triazolo[4,3-a]pyridin-6-ylsulfanyl}benzyl)urea

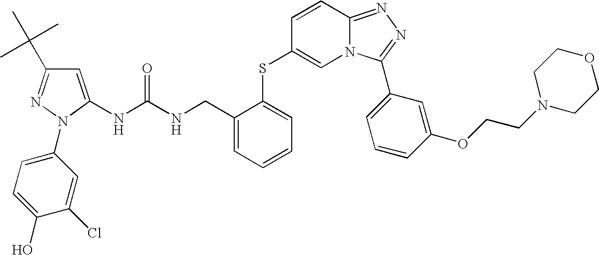

Preparation 50 (314 mg, 362 mmol) in tetrahydrofuran (2 mL) was treated with triethylamine trihydrofluoride (113 mg, 724 μmol) and the solution left to stir at RT for 20 h. The solvent was removed and the mixture taken up in dichloromethane (20 mL), washed with sat. sodium hydrogencarbonate (20 mL), brine (20 mL) and dried (MgSO₄). The material was purified by chromatography (0-5% methanol in dichloromethane+1% ammonia) then crystallised (pyridine-water) to yield pale brown platelets (197 mg, 72%).

¹H NMR (300 MHz, CDCl 3) 8:1.27 (9H, s), 2.60-2.70 (4H, bs), 2.86 (2H, t), 3.70-3.77 (4H, m), 4.16 (2H, t), 4.58 (2H, d), 6.33 (1H, s), 6.57 (1H, d), 6.61-6.68 (1H, bs), 6.89 (2H, bd), 7.00 (1H, dd), 7.12 (1H, d), 7.17 (1H, d), 7.21-7.28 (3>>H, m), 7.30-7.38 (3H, m), 7.46 (1H, d), 7.68 (1H, bs), 7.97 (1H, s).

LCMS: m/z ES 753 [M-H]⁻, RT=2.42 min.

### Example 10

### 1-[5-tert-Butyl-2-(4-chloro-3-hydroxyphenyl)-2H-pyrazol-3-yl]-3-(2-{3-[3-(2-morpholin-4-yl-ethoxy)phenyl]-[1,2,4]triazolo[4,3-a]pyridin-6-ylsulfanyl}benzyl)urea

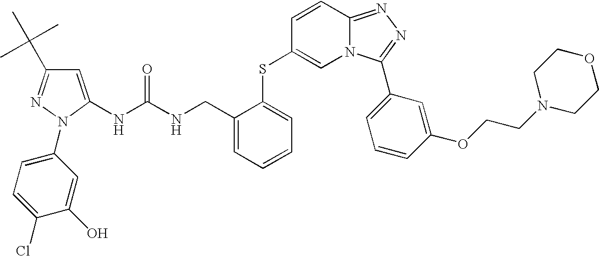

Prepared using the procedure for example 9, using preparation 51 and crystallised (acetone) to yield a pale yellow powder (355 mg, 82%).

¹H NMR (300 MHz, CDCl 3) δ: 1.28 (9H, s), 2.69 (4H, bs), 2.86 (2H, t), 3.72-3.80 (4H, m), 4.14 (2H, t), 4.61 (2H, d), 6.35 (1H, s), 6.75-6.80 (1H, bs), 6.78 (1H, dd), 6.85-6.91 (2H, m), 6.94 (1H, dd), 7.02-7.08 (3H, m), 7.12 (1H, d), 7.22-7.40 (4H, m), 7.49 (1H, d), 7.85 (1H, bs), 7.86 (1H, s).

LRMS: m/z ES 753 [MH]⁺, 751 [MH]

### Example 11

### 1-{2-[3-(5-Chloro-2-hydroxy-phenyl)-[1,2,4]triazolo[4,3-a]pyridin-6-ylsulfanyl]benzyl}-3-[2-(3-dimethylaminomethyl-phenyl)-5-(1,1-dimethyl-2-methylsulfanylethyl)-2H-pyrazol-3-yl]urea

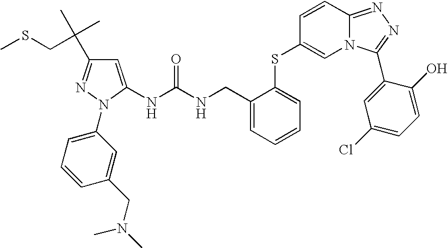

Prepared using the procedure for example 3, using preparation 41 and preparation 15 to yield a white solid (32 mg, 5%)

¹H NMR (300 MHz, DMSO_{d6}) δ: 1.29 (6H, s), 1.97 (3H, s), 2.14 (6H, s), 2.77 (2H, s), 3.42 (2H, s), 4.36 (2H, d), 6.26 (1H, s), 6.91 (1H, t), 6.98-7.04 (1H, m), 7.08-7.14 (1H, m), 7.19-7.44 (10H, m), 7.84 (1H, dd), 8.14-8.17 (1H, m), 8.27 (1H, s), 9.82 (1H, s).

LRMS: m/z APCI/ES 727/729 [MH]⁺, 727/725 [M-H]⁻

The methods described in the above-mentioned examples and preparations can be used to prepare other compounds of the invention. It will be appreciated by those skilled in the art that it may be necessary or desirable at any stage in the synthesis of compounds of the invention to protect one or more sensitive groups in the molecule so as to prevent undesirable side reactions. The skilled person will appreciate that other routes may be equally as practicable.

In another embodiment of the invention, there is provided a compound selected from the following list¹. Said compounds can be prepared using the methods described herein.

### List¹:

1-{5-tert-Butyl-2-[3-(2-dimethylaminoethoxy)phenyl]-2H-pyrazol-3-yl}-3-{2-[3-(5-fluoro-2-hydroxyphenyl)-[1,2,4]triazolo[4,3-a]pyridin-6-ylsulfanyl]benzyl}urea
1-[5-tert-Butyl-2-(3-morpholin-4-ylmethylphenyl)-2H-pyrazol-3-yl]-3-{2-[3-(2-fluoro-5-hydroxyphenyl)-[1,2,4]triazolo[4,3-a]pyridin-6-ylsulfanyl]benzyl}urea
1-[5-tert-Butyl-2-(3-morpholin-4-ylmethylphenyl)-2H-pyrazol-3-yl]-3-{2-[3-(5-fluoro-2-hydroxyphenyl)-[1,2,4]triazolo[4,3-a]pyridin-6-ylsulfanyl]benzyl}urea
1-(3-tert-butyl-1-{4-[2-(dimethylamino)ethoxy]phenyl}-1H-pyrazol-5-yl)-3-(2-{[3-(5-chloro-2-hydroxyphenyl)[1,2,4]triazolo[4,3-a]pyridin-6-yl]thio}benzyl)urea
1-(3-tert-butyl-1-{4-[2-(dimethylamino)ethoxy]phenyl}-1H-pyrazol-5-yl)-3-(2-{[3-(2-chloro-5-hydroxyphenyl)[1,2,4]triazolo[4,3-a]pyridin-6-yl]thio}benzyl)urea
1-(3-tert-butyl-1-{4-[2-(dimethylamino)ethoxy]phenyl}-1H-pyrazol-5-yl)-3-(2-{[3-(3-chloro-4-hydroxyphenyl)[1,2,4]triazolo[4,3-a]pyridin-6-yl]thio}benzyl)urea
1-[3-tert-butyl-1-(3-chloro-4-hydroxyphenyl)-1H-pyrazol-5-yl]-3-{2-[(3-{3-[(4-methylpiperazin-1-yl)methyl]phenyl}[1,2,4]triazolo[4,3-a]pyridin-6-yl)thio]benzyl}urea
1-[3-tert-butyl-1-(4-chloro-3-hydroxyphenyl)-1H-pyrazol-5-yl]-3-[2-({3-[3-(1,3-dihydro-2H-isoindol-2-ylmethyl)phenyl][1,2,4]triazolo[4,3-a]pyridin-6-yl}thio)benzyl]urea
1-[3-tert-butyl-1-(3-fluoro-4-hydroxyphenyl)-1H-pyrazol-5-yl]-3-[2-({3-[3-(3,4-dihydroisoquinolin-2(1H)-ylmethyl)phenyl][1,2,4]triazolo[4,3-a]pyridin-6-yl}thio)benzyl]urea
1-{2-[(3-{4-[(4-benzylpiperazin-1-yl)methyl]phenyl}[1,2,4]triazolo[4,3-a]pyridin-6-yl)thio]benzyl}-3-[3-tert-butyl-1-(3-chloro-4-hydroxyphenyl)-1H-pyrazol-5-yl]urea
1-(2-{([3-(5-chloro-2-hydroxyphenyl)[1,2,4]triazolo[4,3-a]pyridin-6-yl]thio}benzyl)-3-{3-[1-methyl-1-(methylthio)ethyl]-1-[3-(pyrrolidin-1-ylmethyl)phenyl]-1H-pyrazol-5-yl}urea
1-[3-tert-butyl-1-(3-fluoro-4-hydroxyphenyl)-1H-pyrazol-5-yl]-3-[2-({3-[4-(morpholin-4-ylmethyl)phenyl][1,2,4]triazolo[4,3-a]pyridin-6-yl}thio)benzyl]urea
1-[3-tert-butyl-1-(4-fluoro-3-hydroxyphenyl)-1H-pyrazol-5-yl]-3-[2-({3-[4-(morpholin-4-ylmethyl)phenyl][1,2,4]triazolo[4,3-a]pyridin-6-yl}thio)benzyl]urea
1-[3-tert-butyl-1-(3-fluoro-4-hydroxyphenyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-[3-tert-butyl-1-(4-fluoro-3-hydroxyphenyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-{1-(3-chloro-4-hydroxyphenyl)-3-[1-methyl-1-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(4-chloro-3-hydroxyphenyl)-3-[1-methyl-1-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(3-fluoro-4-hydroxyphenyl)-3-[1-methyl-1-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(4-fluoro-3-hydroxyphenyl)-3-[1-methyl-1-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(3-chloro-4-hydroxyphenyl)-3-[1-methyl-1-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{1-(4-chloro-3-hydroxyphenyl)-3-[1-methyl-1-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{1-(3-fluoro-4-hydroxyphenyl)-3-[1-methyl-1-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{1-(4-fluoro-3-hydroxyphenyl)-3-[1-methyl-1-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{1-(3-chloro-4-hydroxyphenyl)-3-[1,1-dimethyl-2-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(4-chloro-3-hydroxyphenyl)-3-[1,1-dimethyl-2-(methylthio)ethyl]-1H-pyrazol-5-yl}-3-[2-({3-[4-(morpholin-4-ylmethyl)phenyl][1,2,4]triazolo[4,3-a]pyridin-6-yl}thio)benzyl]urea
1-{3-[1,1-dimethyl-2-(methylthio)ethyl]-1-(3-fluoro-4-hydroxyphenyl)-1H-pyrazol-5-yl}-3-[2-({3-[4-(morpholin-4-ylmethyl)phenyl][1,2,4]triazolo[4,3-a]pyridin-6-yl}thio)benzyl]urea
1-{3-[1,1-dimethyl-2-(methylthio)ethyl]-1-(4-fluoro-3-hydroxyphenyl)-1H-pyrazol-5-yl}-3-[2-({3-[4-(morpholin-4-ylmethyl)phenyl][1,2,4]triazolo[4,3-a]pyridin-6-yl}thio)benzyl]urea
1-{1-(3-chloro-4-hydroxyphenyl)-3-[1,1-dimethyl-2-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{1-(4-chloro-3-hydroxyphenyl)-3-[1,1-dimethyl-2-(methylthio)ethyl]-1H-pyrazol-5-yl}-3-[2-({3-[3-(2-morpholin-4-ylethoxy)phenyl][1,2,4]triazolo[4,3-a]pyridin-6-yl}thio)benzyl]urea
1′-{3-[1,1-dimethyl-2-(methylthio)ethyl]-1-(3-fluoro-4-hydroxyphenyl)-1H-pyrazol-5-yl}-3-[2-({3-[3-(2-morpholin-4-ylethoxy)phenyl][1,2,4]triazolo[4,3-a]pyridin-6-yl}thio)benzyl]urea
1-{3-[1,1-dimethyl-2-(methylthio)ethyl]-1-(4-fluoro-3-hydroxyphenyl)-1H-pyrazol-5-yl}-3-[2-({3-[3-(2-morpholin-4-ylethoxy)phenyl][1,2,4]triazolo[4,3-a]pyridin-6-yl}thio)benzyl]urea
1-[1-(3-chloro-4-hydroxyphenyl)-3-(1,1-dimethylpropyl)-1H-pyrazol-5-yl]-3-[2-({3-[4-(morpholin-4-ylmethyl)phenyl][1,2,4]triazo[4,3-a]pyridin-6-yl}thio)benzyl]urea
1-[1-(4-chloro-3-hydroxyphenyl)-3-(1,1-dimethylpropyl)-1H-pyrazol-5-yl]-3-[2-({3-[4-(morpholin-4-ylmethyl)phenyl][1,2,4]triazolo[4,3-a]pyridin-6-yl}thio)benzyl]urea
1-[3-(1,1-dimethylpropyl)-1-(3-fluoro-4-hydroxyphenyl)-1H-pyrazol-5-yl]-3-[2-({3-[4-(morpholin-4-ylmethyl)phenyl][1,2,4]triazolo[4,3-a]pyridin-6-yl}thio)benzyl]urea
1-[3-(1,1-dimethylpropyl)-1-(4-fluoro-3-hydroxyphenyl)-1H-pyrazol-5-yl]-3-[2-({3-[4-(morpholin-4-ylmethyl)phenyl][1,2,4]triazolo[4,3-a]pyridin-6-yl}thio)benzyl]urea
1-[1-(3-chloro-4-hydroxyphenyl)-3-(1,1-dimethylpropyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-[1-(4-chloro-3-hydroxyphenyl)-3-(1,1-dimethylpropyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-[3-(1,1-dimethylpropyl)-1-(3-fluoro-4-hydroxyphenyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-[3-(1,1-dimethylpropyl)-1-(4-fluoro-3-hydroxyphenyl)-1H-pyrazol-5-yl]-3-[2-({3-[3-(2-morpholin-4-ylethoxy)phenyl][1,2,4]triazolo[4,3-a]pyridin-6-yl}thio)benzyl]urea
1-(3-tert-butyl-1-{3-[(dimethylamino)methyl]phenyl}-1H-pyrazol-5-yl)-3-(2-{[3-(2-fluoro-5-hydroxyphenyl)[1,2,4]triazolo[4,3-a]pyridin-6-yl]thio}benzyl)urea
1-(3-tert-butyl-1-{3-[2-(dimethylamino)ethoxy]phenyl}-1H-pyrazol-5-yl)-3-(2-{[3-(2-fluoro-5-hydroxyphenyl)[1,2,4]triazolo[4,3-a]pyridin-6-yl]thio}benzyl)urea
1-{3-tert-butyl-1-[3-(pyrrolidin-1-ylmethyl)phenyl]-1H-pyrazol-5-yl}-3-(2-{[3-(2-fluoro-5-hydroxyphenyl)[1,2,4]triazolo[4,3-a]pyridin-6-yl]thio}benzyl)urea
1-{3-tert-butyl-1-[3-(morpholin-4-ylmethyl)phenyl]-1H-pyrazol-5-yl}-3-(2-{[3-(2-fluoro-5-hydroxyphenyl)[1,2,4]tri azolo[4,3-a]pyridin-6-yl]thio}benzyl)urea
1-(3-tert-butyl-1-{3-[(dimethylamino)methyl]phenyl}-1H-pyrazol-5-yl)-3-(2-{[3-(5-fluoro-2-hydroxyphenyl)[1,2,4]triazolo[4,3-a]pyridin-6-yl]thio}benzyl)urea
1-(3-tert-butyl-1-{3-[2-(dimethylamino)ethoxy]phenyl}-1H-pyrazol-5-yl)-3-(2-{[3-(5-fluoro-2-hydroxyphenyl)[1,2,4]triazolo[4,3-a]pyridin-6-yl]thio}benzyl)urea
1-{3-tert-butyl-1-[3-(pyrrolidin-1-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-{3-tert-butyl-1-[3-(morpholin-4-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-{3-tert-butyl-1-[3-(pyrrolidin-1-ylmethyl)phenyl]-1H-pyrazol-5-yl}-3-(2-{[3-(5-chloro-2-hydroxyphenyl)[1,2,4]triazolo[4,3-a]pyridin-6-yl]thio}benzyl)urea
1-(3-tert-butyl-1-{3-[2-(dimethylamino)ethoxy]phenyl}-1H-pyrazol-5-yl)-3-(2-{[3-(2-chloro-5-hydroxyphenyl)[1,2,4]triazolo[4,3-a]pyridin-6-yl]thio}benzyl)urea
1-{3-tert-butyl-1-[3-(pyrrolidin-1-ylmethyl)phenyl]-1H-pyrazol-5-yl}-3-(2-{[3-(2-chloro-5-hydroxyphenyl)[1,2,4]triazolo[4,3-a]pyridin-6-yl]thio}benzyl)urea
1-(1-{3-[(dimethylamino)methyl]phenyl}-3-[1-methyl-1-(methylthio)ethyl]-1H-pyrazol-5-yl)-3-(2-{[3-(2-fluoro-5-hydroxyphenyl)[1,2,4]triazolo[4,3-a]pyridin-6-yl]thio}benzyl)urea
1-(1-{3-[2-(dimethylamino)ethoxy]phenyl}-3-[1-methyl-1-(methylthio)ethyl]-1H-pyrazol-5-yl)-3-(2-{[3-(2-fluoro-5-hydroxyphenyl)[1,2,4]triazolo[4,3-a]pyridin-6-yl]thio}benzyl)urea
1-(2-{[3-(2-fluoro-5-hydroxyphenyl)[1,2,4]triazolo[4,3-a]pyridin-6-yl]thio}benzyl)-3-{3-[1-methyl-1-(methylthio)ethyl]-1-[3-(pyrrolidin-1-ylmethyl)phenyl]-1H-pyrazol-5-yl}urea
1-(2-{[3-(2-fluoro-5-hydroxyphenyl)[1,2,4]triazolo[4,3-a]pyridin-6-yl]thio}benzyl)-3-{3-[1-methyl-1-(methylthio)ethyl]-1-[3-(morpholin-4-ylmethyl)phenyl]-1H-pyrazol-5-yl}urea
1-(1-{3-[(dimethylamino)methyl]phenyl}-3-[1-methyl-1-(methylthio)ethyl]-1H-pyrazol-5-yl)-3-(2-{[3-(5-fluoro-2-hydroxyphenyl)[1,2,4]triazolo[4,3-a]pyridin-6-yl]thio}benzyl)urea
1-(1-{3-[2-(dimethylamino)ethoxy]phenyl}-3-[1-methyl-1-(methylthio)ethyl]-1H-pyrazol-5-yl)-3-(2-{[3-(5-fluoro-2-hydroxyphenyl)[1,2,4]triazolo[4,3-a]pyridin-6-yl]thio}benzyl)urea
1-(2-{[3-(5-fluoro-2-hydroxyphenyl)[1,2,4]triazolo[4,3-a]pyridin-6-yl]thio}benzyl)-3-{3-[1-methyl-1-(methylthio)ethyl]-1-[3-(pyrrolidin-1-ylmethyl)phenyl]-1H-pyrazol-5-yl}urea
1-(2-{[3-(5-fluoro-2-hydroxyphenyl)[1,2,4]triazolo[4,3-a]pyridin-6-yl]thio}benzyl)-3-{3-[1-methyl-1-(methylthio)ethyl]-1-[3-(morpholin-4-ylmethyl)phenyl]-1H-pyrazol-5-yl}urea
1-(2-{[3-(5-chloro-2-hydroxyphenyl)[1,2,4]triazolo[4,3-a]pyridin-6-yl]thio}benzyl)-3-(1-{3-[(dimethylamino)methyl]phenyl}-3-[1-methyl-1-(methylthio)ethyl]-1H-pyrazol-5-yl)urea
1-(2-{[3-(5-chloro-2-hydroxyphenyl)[1,2,4]triazolo[4,3-a]pyridin-6-yl]thio}benzyl)-3-(1-{3-[2-(dimethylamino)ethoxy]phenyl}-3-[1-methyl-1-(methylthio)ethyl]-1H-pyrazol-5-yl)urea
1-(2-{[3-(5-chloro-2-hydroxyphenyl)[1,2,4]triazolo[4,3-a]pyridin-6-yl]thio}benzyl)-3-{3-[1-methyl-1-(methylthio)ethyl]-1-[3-(pyrrolidin-1-ylmethyl)phenyl]-1H-pyrazol-5-yl}urea
1-(2-{[3-(5-chloro-2-hydroxyphenyl)[1,2,4]triazolo[4,3-a]pyridin-6-yl]thio}benzyl)-3-{3-[1-methyl-1-(methylthio)ethyl]-1-[3-(morpholin-4-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-(1-{3-[(dimethylamino)methyl]phenyl}-3-[1-methyl-1-(methylthio)ethyl]-1H-pyrazol-5-yl)urea
1-(2-{[3-(2-chloro-5-hydroxyphenyl)[1,2,4]triazolo[4,3-a]pyridin-6-yl]thio}benzyl)-3-(1-{3-[2-(dimethylamino)ethoxy]phenyl}-3-[1-methyl-1-(methylthio)ethyl]-1H-pyrazol-5-yl)urea
1-(2-{[3-(2-chloro-5-hydroxyphenyl)[1,2,4]triazolo[4,3-a]pyridin-6-yl]thio}benzyl)-3-{3-[1-methyl-1-(methylthio)ethyl]-1-[3-(pyrrolidin-1-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-{3-[1-methyl-1-(methylthio)ethyl]-1-[3-(morpholin-4-ylmethyl)phenyl]-1H-pyrazol-5-yl}urea
1-(1-{3-[(dimethylamino)methyl]phenyl}-3-[1,1-dimethyl-2-(methylthio)ethyl]-1H-pyrazol-5-yl)-3-(2-{[3-(2-fluoro-5-hydroxyphenyl)[1,2,4]triazolo[4,3-a]pyridin-6-yl]thio}benzyl)urea
1-(1-{3-[2-(dimethylamino)ethoxy]phenyl}-3-[1,1-dimethyl-2-(methylthio)ethyl]-1H-pyrazol-5-yl)-3-(2-{[3-(2-fluoro-5-hydroxyphenyl)[1,2,4]triazolo[4,3-a]pyridin-6-yl]thio}benzyl)urea
1-{3-[1,1-dimethyl-2-(methylthio)ethyl]-1-[3-(pyrrolidin-1-ylmethyl)phenyl]-1H-pyrazol-5-yl}-3-(2-{[3-(2-fluoro-5-hydroxyphenyl)[1,2,4]triazolo[4,3-a]pyridin-6-yl]thio}benzyl)urea
1-{3-[1,1-dimethyl-2-(methylthio)ethyl]-1-[3-(morpholin-4-ylmethyl)phenyl]-1H-pyrazol-5-yl}-3-(2-{[3-(2-fluoro-5-hydroxyphenyl)[1,2,4]triazolo[4,3-a]pyridin-6-yl]thio}benzyl)urea
1-(1-{3-[(dimethylamino)methyl]phenyl}-3-[1,1-dimethyl-2-(methylthio)ethyl]-1H-pyrazol-5-yl)-3-(2-{[3-(5-fluoro-2-hydroxyphenyl)[1,2,4]triazolo[4,3-a]pyridin-6-yl]thio}benzyl)urea
1-(1-{3-[2-(dimethylamino)ethoxy]phenyl}-3-[1,1-dimethyl-2-(methylthio)ethyl]-1H-pyrazol-5-yl)-3-(2-{[3-(5-fluoro-2-hydroxyphenyl)[1,2,4]triazolo[4,3-a]pyridin-6-yl]thio}benzyl)urea
1-{3-[1,1-dimethyl-2-(methylthio)ethyl]-1-[3-(pyrrolidin-1-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-{3-[1,1-dimethyl-2-(methylthio)ethyl]-1-[3-(morpholin-4-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-(2-{[3-(5-chloro-2-hydroxyphenyl)[1,2,4]triazolo[4,3-a]pyridin-6-yl]thio}benzyl)-3-(1-{3-[2-(dimethylamino)ethoxy]phenyl}-3-[1,1-dimethyl-2-(methylthio)ethyl]-1H-pyrazol-5-yl)urea
1-(2-{[3-(5-chloro-2-hydroxyphenyl)[1,2,4]triazolo[4,3-a]pyridin-6-yl]thio}benzyl)-3-{3-[1,1-dimethyl-2-(methylthio)ethyl]-1-[3-(pyrrolidin-1-ylmethyl)phenyl]-1H-pyrazol-5-yl}urea
1-(2-{[3-(5-chloro-2-hydroxyphenyl)[1,2,4]triazolo[4,3-a]pyridin-6-yl]thio}benzyl)-3-{3-[1,1-dimethyl-2-(methylthio)ethyl]-1-[3-(morpholin-4-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-(1-{3-[2-(dimethylamino)ethoxy]phenyl}-3-[1,1-dimethyl-2-(methylthio)ethyl]-1H-pyrazol-5-yl)urea
1-(2-{[3-(2-chloro-5-hydroxyphenyl)[1,2,4]triazolo[4,3-a]pyridin-6-yl]thio}benzyl)-3-{3-[1,-dimethyl-2-(methylthio)ethyl]-1-[3-(pyrrolidin-1-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-{3-[1,1-dimethyl-2-(methylthio)ethyl]-1-[3-(morpholin-4-ylmethyl)phenyl]-1H-pyrazol-5-yl}urea
1-[1-{3-[(dimethylamino)methyl]phenyl}-3-(1,1-dimethylpropyl)-1H-pyrazol-5-yl]-3-(2-{[3-(2-fluoro-5-hydroxyphenyl)[1,2,4]triazolo[4,3-a]pyridin-6-yl]thio}benzyl)urea
1-[1-{3-[2-(dimethylamino)ethoxy]phenyl}-3-(1,1-dimethylpropyl)-1H-pyrazol-5-yl]-3-(2-{[3-(2-fluoro-5-hydroxyphenyl)[1,2,4]triazolo[4,3-a]pyridin-6-yl]thio}benzyl)urea
1-{3-(1,1-dimethylpropyl)-1-[3-(pyrrolidin-1-ylmethyl)phenyl]-1H-pyrazol-5-yl}-3-(2-{[3-(2-fluoro-5-hydroxyphenyl)[1,2,4]triazolo[4,3-a]pyridin-6-yl]thio}benzyl)urea
1-{3-(1,1-dimethylpropyl)-1-[3-(morpholin-4-ylmethyl)phenyl]-1H-pyrazol-5-yl}-3-(2-{[3-(2-fluoro-5-hydroxyphenyl)[1,2,4]triazolo[4,3-a]pyridin-6-yl]thio}benzyl)urea
1-[1-{3-[(dimethylamino)methyl]phenyl}-3-(1,1-dimethylpropyl)-1H-pyrazol-5-yl]-3-(2-{[3-(5-fluoro-2-hydroxyphenyl)[1,2,4]triazolo[4,3-a]pyridin-6-yl]thio}benzyl)urea
1-[1-{3-[2-(dimethylamino)ethoxy]phenyl}-3-(1,1-dimethylpropyl)-1H-pyrazol-5-yl]-3-(2-{[3-(5-fluoro-2-hydroxyphenyl)[1,2,4]triazolo[4,3-a]pyridin-6-yl]thio}benzyl)urea
1-{3-(1,1-dimethylpropyl)-1-[3-(pyrrolidin-1-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-{3-(1,1-dimethylpropyl)-1-[3-(morpholin-4-ylmethyl)phenyl]-1H-pyrazol-5-yl}-3-(2-{[3-(5-fluoro-2-hydroxyphenyl)[1,2,4]triazolo[4,3-a]pyridin-6-yl]thio}benzyl)urea
1-(2-{[3-(5-chloro-2-hydroxyphenyl)[1,2,4]triazolo[4,3-a]pyridin-6-yl]thio}benzyl)-3-[1-{3-[(dimethylamino)methyl]phenyl}-3-(1,1-dimethylpropyl)-1H-pyrazol-5-yl]urea
1-(2-{[3-(5-chloro-2-hydroxyphenyl)[1,2,4]triazolo[4,3-a]pyridin-6-yl]thio}benzyl)-3-[1-{3-[2-(dimethylamino)ethoxy]phenyl}-3-(1,1-dimethylpropyl)-1H-pyrazol-5-yl]urea
1-(2-{[3-(5-chloro-2-hydroxyphenyl)[1,2,4]triazolo[4,3-a]pyridin-6-yl]thio}benzyl)-3-{3-(1,1-dimethylpropyl)-1-[3-(pyrrolidin-1-ylmethyl)phenyl]-1H-pyrazol-5-yl}urea
1-(2-{[3-(5-chloro-2-hydroxyphenyl)[1,2,4]triazolo[4,3-a]pyridin-6-yl]thio}benzyl)-3-{3-(1,1-dimethylpropyl)-1-[3-(morpholin-4-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-[1-{3-[(dimethylamino)methyl]phenyl}-3-(1,1-dimethylpropyl)-1H-pyrazol-5-yl]urea
1-(2-{[3-(2-chloro-5-hydroxyphenyl)[1,2,4]triazolo[4,3-a]pyridin-6-yl]thio}benzyl)-3-[1-{3-[2-(dimethylamino)ethoxy]phenyl}-3-(1,1-dimethylpropyl)-1H-pyrazol-5-yl]urea
1-(2-{[3-(2-chloro-5-hydroxyphenyl)[1,2,4]triazolo[4,3-a]pyridin-6-yl]thio}benzyl)-3-{3-(1,1-dimethylpropyl)-1-[3-(pyrrolidin-1-ylmethyl)phenyl]-1H-pyrazol-5-yl}urea
1-(2-{[3-(2-chloro-5-hydroxyphenyl)[1,2,4]triazolo[4,3-a]pyridin-6-yl]thio}benzyl)-3-{3-(1,1-dimethylpropyl)-1-[3-(morpholin-4-ylmethyl)phenyl]-1H-pyrazol-5-yl}urea
1-{3-tert-butyl-1-[3-(thiomorpholin-4-ylmethyl)phenyl]-1H-pyrazol-5-yl}-3-(2-{[3-(5-chloro-2-hydroxyphenyl)[1,2,4]triazolo[4,3-a]pyridin-6-yl]thio}benzyl)urea

1. A compound of formula (Ia): 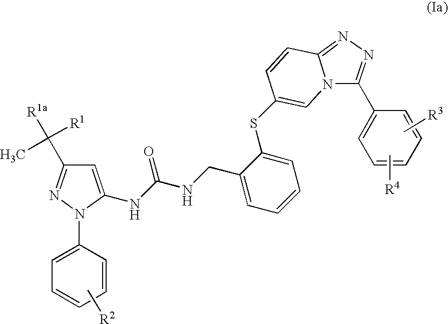
or a pharmaceutically acceptable salt, solvate or hydrate thereof, wherein
R¹ is CH₃, SCH₃, SCH₂CH₃, CH₂CH₃, H or CH₂SCH₃;
R^{1a} is CH₃ or CH₂CH₃;
R² is 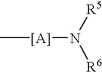
wherein A is selected from —O—(CH₂)ₓ— where x is 2 or 3, and —(CH₂)_{y}— wherein y is
1, 2 or 3;
R⁵ and R⁶ are each independently selected from methyl, ethyl and propyl, or together with the nitrogen atom to which they are attached form a pyrrolidinyl, morpholinyl, thiomorpholinyl, piperidinyl or piperazinyl ring;
and one of R³ and R⁴ is hydroxy, and the other is selected from chloro and fluoro;
or a compound of formula (Ib): 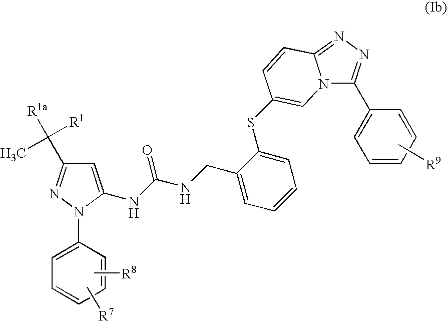
or a pharmaceutically acceptable salt thereof, wherein
one of R⁷ and R⁸ is hydroxy, and the other is selected from chloro and fluoro;
R⁹ is in the 3 or 4 position of the phenyl ring, and is 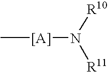
wherein A is as defined above for formula (Ia), and R¹⁰ and R¹¹ are each independently selected from methyl, ethyl, propyl, benzyl and phenylethyl, or together with the nitrogen atom to which they are attached R¹⁰ and R¹¹ form a pyrrolidinyl, piperidinyl, morpholinyl, thiomorpholinyl or piperazinyl ring, wherein said piperazinyl ring is optionally substituted at the 4 position with methyl, ethyl, propyl or benzyl, and wherein said pyrrolidinyl and piperidinyl are each optionally fused with a phenyl ring;
and R¹ and R^{1a} are as defined above for formula (Ia).

2. A compound of claim 1 or a pharmaceutically acceptable salt, solvate or hydrate thereof wherein R¹ is CH₃, SCH₃, CH₂SCH₃ or CH₂CH₃.

3. A compound of claim 1 or a Pharmaceutically acceptable salt, solvate or hydrate thereof wherein R^{1a} is CH₃.

4. A compound of claim 1 or a pharmaceutically acceptable salt, solvate or hydrate thereof wherein A is ethoxy or methyl.

5. A compound of claim 1 or a pharmaceutically acceptable salt, solvate or hydrate thereof wherein R⁵ and R⁶ are both methyl, or together with the nitrogen atom to which they are attached form a pyrrolidinyl or a morpholinyl ring.

6. A compound, of claim 1 or a pharmaceutically acceptable salt, solvate or hydrate thereof wherein R² is dimethylaminoethoxy, dimethylaminomethyl or morpholin-4-ylmethyl.

7. A compound of claim 1 or a pharmaceutically acceptable salt, solvate or hydrate thereof wherein R² is in the 3-position of the phenyl ring.

8. A compound of claim 1 or a pharmaceutically acceptable salt, solvate or hydrate thereof wherein one of R³ and R⁴ is hydroxy and the other is chloro.

9. A compound of claim 1 or a pharmaceutically acceptable salt, solvate or hydrate thereof wherein R³ and R⁴ are in the 2- and 5-positions of the phenyl ring.

10. A compound of claim 1 or a pharmaceutically acceptable salt, solvate or hydrate thereof wherein one of R⁷ and R⁸ is hydroxy, and the other is chloro.

11. A compound of claim 1 or a pharmaceutically acceptable salt, solvate or hydrate thereof wherein R⁷ and R⁸ are in the 3- and 4-positions of the phenyl ring.

12. A compound of claim 1 or a pharmaceutically acceptable salt, solvate or hydrate thereof wherein R⁹ is morpholin-4-ylmethyl or morpholin-4-ylethoxy.

13. A compound, of claim 12 or a pharmaceutically acceptable salt, solvate or hydrate thereof wherein R⁹ is 4-(morpholin-4-ylmethyl) or 3-(morpholin-4-ylethoxy).

14-15. (canceled)

16. A compound of claim 1 wherein the pharmaceutically acceptable salt is selected from acetate, mesylate, fumarate, hydrochloride/chloride, hydrobromide/bromide, bisulphate/sulphate, D-Tartrate, L-Tartrate, isethionate and xinafoate.

17-23. (canceled)

24. A pharmaceutical composition comprising a compound of claim 1 or a pharmaceutically acceptable salt, solvate or hydrate thereof and a pharmaceutically acceptable diluent, carrier or adjuvant.

25. A method of treating a disease, disorder, or condition in a mammal, said method comprising administering to a said mammal (a) an effective amount of a compound of claim 1 or a pharmaceutically acceptable salt thereof or (b) a pharmaceutical composition comprising a compound of claim 1 or a pharmaceutically acceptable salt thereof and a pharmaceutically acceptable diluent, carrier or adjuvant, wherein said disease, disorder, or condition is selected from asthma, chronic or acute bronshoconstriction, bronchitis, small airways obstruction, emphysema, obstructive or inflammatory airways disease, acute lung injury and bronchiectasis.

26. (canceled)

27. A method of claim 25 wherein said asthma is selected from atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

28. A method of claim 25 wherein said bronchitis is selected from acute bronchitis, chronic bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis.

29. A method of claim 25 wherein said bronchiectasis is selected from cylindric bronchiectasis, sacculated bronchiectasis, fusiform bronchiectasis, capillary bronchiectasis, cystic bronchiectasis, dry bronchiectasis and follicular bronchiectasis.′

30. A method of claim 25 wherein said obstructive or inflammatory airways disease is selected from chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

31. A method of claim 25 wherein said disease, disorder, or condition is asthma or chronic obstructive pulmonary disease (COPD).
